(19) United States
(12) Patent Application Publication
(10) Pub. No.: US 2025/0161687 A1
(43) Pub. Date: May 22, 2025

(54) METHODS FOR POSITIONING PATIENT TREATMENT SYSTEMS TO SENSE CARDIAC DEPOLARIZATION AND/OR STIMULATE AFFERENT FIBERS, AND ASSOCIATED DEVICES AND SYSTEMS

(71) Applicant: Barologics, Inc., Carver, MN (US)

(72) Inventors: Molly Wade, Carver, MN (US); Eric Lovett, Mendota Heights, MN (US); Adam W. Cates, Atlanta, GA (US); David Opperman, Denver, CO (US); Mark Gryzwa, Woodbury, MN (US)

(21) Appl. No.: 18/949,178

(22) Filed: Nov. 15, 2024

Related U.S. Application Data
(60) Provisional application No. 63/599,952, filed on Nov. 16, 2023.

(51) Int. Cl. A61N 1/36

(52) U.S. Cl. CPC A61N 1/36139

(57) ABSTRACT

Patient treatment systems and methods for sensing cardiac depolarization and/or stimulating the carotid sinus nerve are disclosed herein. In some embodiments, a method of implanting a signal delivery device to deliver an electrical signal to a patient includes identifying a target region of a blood vessel of the patient; forming an opening on a side of the target region to partially separate periadventitial tissue surrounding the blood vessel, including fibers associated with baroreceptors and/or the carotid sinus nerve of the patient, from the underlying the blood vessel; positioning a first region of the signal delivery device at least partially through the opening between the blood vessel and the periadventitial tissue; and positioning a second region of the signal delivery device at least partially over the first region such that the periadventitial tissue is between the first region and the second region.

### CROSS-REFERENCE TO RELATED APPLICATIONS

The present application is related to U.S. patent application Ser. No. 18/333,416, filed Jun. 12, 2023, and titled “PATIENT TREATMENT SYSTEMS FOR SENSING CARDIAC DEPOLARIZATION AND/OR STIMULATING THE CAROTID SINUS NERVE, AND ASSOCIATED DEVICES AND METHODS,” and claims the benefit of U.S. Provisional Patent Application No. 63/599,952, filed Nov. 16, 2023, and titled “METHODS FOR POSITIONING PATIENT TREATMENT SYSTEMS TO SENSE CARDIAC DEPOLARIZATION AND/OR STIMULATE AFFERENT FIBERS, AND ASSOCIATED DEVICES AND SYSTEMS,” the disclosures of which are each incorporated herein by reference in their entirety.

### TECHNICAL FIELD

This disclosure relates to methods for positioning patient treatment systems to sense cardiac depolarization and/or stimulate afferent fibers, and associated devices and systems.

### BACKGROUND

Millions of patients worldwide suffer from cardiovascular diseases, such as hypertension (i.e., high blood pressure) and heart failure. Many different pharmaceutical and medical device treatments have been developed to treat hypertension and heart failure, but many of these treatments have been either completely ineffective or at least ineffective in large subsets of patients. For example, approximately one in ten people with high blood pressure are treatment resistant, in that pharmaceuticals do not help to reduce their blood pressure. Approximately one hundred million people worldwide suffer from treatment resistant high blood pressure, and these patients are three times more likely to suffer from a cardiovascular event, such as a heart attack, compared to patients whose blood pressure can be controlled with medications.

Several different medical devices and procedures have been tried to treat drug resistant high blood pressure. One example is a procedure in which a catheter is threaded into the arteries leading to the kidneys, and radiofrequency energy is applied to the vessel wall to denervate the small nerves surrounding the arteries. Another example is an implantable stimulator for stimulating baroreceptors in the neck by applying energy to the wall of the carotid artery. Unfortunately, these devices and procedures have not been proven to be as effective as desired. Currently, hundreds of millions of patients suffer from currently untreatable high blood pressure, which very often leads to serious cardiovascular consequences. Unfortunately, other serious health conditions, such as congestive heart failure and kidney failure, have similar stories. Therefore, a need exists for improved devices, systems, and methods for treating hypertension, heart failure. and/or other cardiovascular conditions.

In addition to the need for improved devices, implanting such devices at target zones around the carotid sinus can be difficult. For example, the carotid sinus nerve (CSN) is small and fragile, and not entirely consolidated (i.e., the fibers comprising the nerve structure may be spatially dispersed, particularly in the region close to the carotid bifurcation). Additionally, the surrounding anatomy/tissue directly overlays the CSN, making incisions and initial exposure of the carotid bifurcation problematic. Accordingly, a further need exists for improved methods to identify the target zone and implant the device without damaging the local anatomy.

### BRIEF DESCRIPTION OF THE DRAWINGS

Features, aspects, and advantages of the presently disclosed technology may be better understood with regard to the following drawings.

FIGS. 1A and 1B are anatomical illustrations of the carotid sinus and surrounding nerves in the anatomical area.

FIGS. 2A-2D are anatomical illustrations showing different patterns of a vagus nerve around the carotid sinus.

FIG. 3 is a partially schematic illustration of a patient treatment system implanted at a carotid sinus nerve (CSN) of a patient, in accordance with embodiments of the present technology.

FIG. 4 is a partially schematic illustration of a carotid sinus nerve stimulation (CSNS) neuromonitoring system, in accordance with embodiments of the present technology.

FIGS. 5A and 5B are partially schematic illustrations of various embodiments of a patient treatment system, in accordance with embodiments of the present technology.

FIGS. 6A and 6B are partially schematic illustrations of various embodiments of signal delivery devices, configured in accordance with embodiments of the present technology.

FIG. 7 is a flow diagram of a method for positioning a patient treatment system, in accordance with embodiments of the present technology.

FIGS. 8A-8D are illustrations of various stages of the method of FIG. 7.

FIG. 9A is a cross-section taken along section line 9A-9A in FIG. 8A.

FIG. 9B is a cross-section taken along section line 9B-9B in FIG. 8B.

FIG. 10A is a cross-section taken along section line 10A-10A in FIG. 9A.

FIG. 10B is a cross-section taken along section line 10B-10B in FIG. 9B.

FIGS. 11A-11D are images of tissue illustrating representative implant positions for a signal delivery device, in accordance with embodiments of the present technology.

FIG. 12 is a partially schematic illustration of a patient treatment system implanted in a patient, in accordance with embodiments, of the present technology.

FIG. 13 is a flow diagram of a method for positioning a signal delivery device, in accordance with embodiments of the present technology.

FIG. 14 is a flow diagram of a method for positioning a signal delivery device, in accordance with embodiments of the present technology.

A person skilled in the relevant art will understand that the features shown in the drawings are for purposes of illustration, and variations, including different and/or additional features and arrangements thereof, are possible.

### DETAILED DESCRIPTION

### I. Overview

Embodiments of the present disclosure relate to patient treatment systems for sensing cardiac depolarization events and/or providing time-varying therapy to afferent fibers associated with baroreceptors and/or CSN afferent fibers of patients based on one or more physiological parameters (e.g., heart rate, R-R wave interval, blood pressure, etc.) obtained from the patients. It is generally known that baroreceptors at the carotid sinus contain stretch receptors that respond to cardiac depolarization, including biological activity caused by cardiac depolarization (e.g., contraction of the left ventricle and one or more arterial pulses resulting therefrom), by relaying associated signals to the brain. In patients with hypertension, the mechanism for relaying such signals may be dysfunctional and therein limit the natural ability of the patients to regulate heart rate. Current devices that attempt to provide therapy to patients to treat hypertension via stimulation provide tonic therapy (i.e., a set frequency, amplitude, pulse width, etc.) that does not change based on patient activity, and that is provided to anatomy that has a less effective or desirable response to therapy.

Embodiments of the present disclosure address at least some of the above-described issues for patients with hypertension. For example, embodiments of the present disclosure utilize neuromodulation of the CSN to alter a patient's abnormal response and therein lower blood pressure. As disclosed herein, patient treatment systems of the present technology can map a patient's tissue by sensing cardiac depolarization and the associated electrical and/or acoustic signals, and position lead electrodes (e.g., in a spiral cuff arrangement, a flat cuff or “book” arrangement, a single surface array/electrode arrangement, etc.) of the patient treatment system at least substantially proximate the CSN afferent fibers. Once positioned, the patient treatment system can determine one or more physiological parameters of the patient and provide stimulation to the patient based at least in part on the one or more physiological parameters. Additionally, due in part to the ability to sense a pulse, such as an arterial pulse and/or a cardiac depolarization, the patient treatment system can provide neuromodulation pulses having stimulation characteristics (e.g., frequency, amplitude, pulse width, delay, etc.) that in some embodiments mimic a natural and desirable baroreceptor response (e.g., the response of patients without hypertension). In doing so, embodiments of the present technology can automatically (e.g., without user input) adjust stimulation parameters based at least in part on a patient's activity, and therein provide non-tonic therapy that is not generally diluted by a patient's activity. For these and other reasons disclosed herein, embodiments of the present technology offer patient therapy that is an improvement over existing devices and methods.

As noted above, methods for installing the signal delivery device at the CSN or target zone can be difficult due to the small and fragile nature of the CSN and the varying location of target fibers from patient to patient. Embodiments of the present technology attempt to address this issue by utilizing methods for identifying the target tissue and carotid bifurcation, dissecting the target tissue, and placing the signal delivery device at the target tissue. In some embodiments, the patient treatment systems are installed using the capabilities of the system itself (e.g., electrodes of the signal delivery device) and/or with the assistance of external systems such as advanced neuromonitoring systems, external devices, controllers, and physician programmers.

In the Figures, identical reference numbers identify generally similar, and/or identical, elements. Many of the details, dimensions, and other features shown in the Figures are merely illustrative of particular embodiments of the disclosed technology. Accordingly, other embodiments can have other details, dimensions, and features without departing from the spirit or scope of the disclosure. In addition, those of ordinary skill in the art will appreciate that further embodiments of the various disclosed technologies can be practiced without several of the details described below.

### II. Anatomy of the Carotid Sinus Nerve (CSN) and Vagus Nerve

Disclosed herein are methods, devices, and systems for sensing cardiac depolarization and/or stimulating nerves to treat hypertension, coronary heart disease, heart failure, kidney disease, and/or any of a number of other disease states in humans or animals. Although the following description will focus on the treatment of drug-resistant hypertension or high blood pressure, the aspects and principles described below may be used to treat, or be adapted for use to treat, several cardiovascular or other conditions. Therefore, despite the focus of the following description on one disease state, the scope of this disclosure and the methods, devices, and systems described herein are not limited to any one disease or condition.

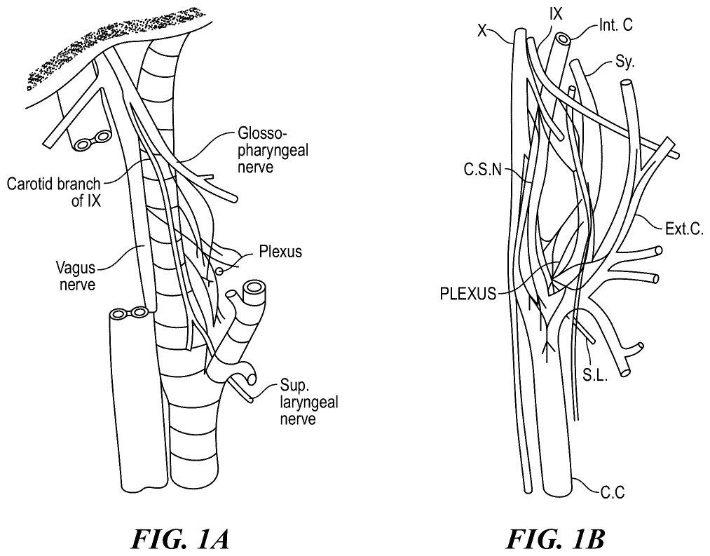

FIGS. 1A and 1B are anatomical illustrations of the carotid sinus and nerves in the surrounding anatomical area. Referring to FIGS. 1A and 1B together, there are two branches of the CSN arising from its origin in the main trunk of the glossopharyngeal nerve IX (i.e., cranial nerve IX). (The vagus nerve, or cranial nerve X, is labeled “X” in FIG. 1B.) One branch of the CSN courses along the anteromedial aspect of the internal carotid artery (ICA) (or “Int. C” in FIG. 1B), terminating in the bifurcation of the carotid sinus and plexus lying posterior and medial to the ICA in the bifurcation of the common carotid artery (CCA) (or “CC” in FIG. 1B). The other branch terminates in the plexus directly.

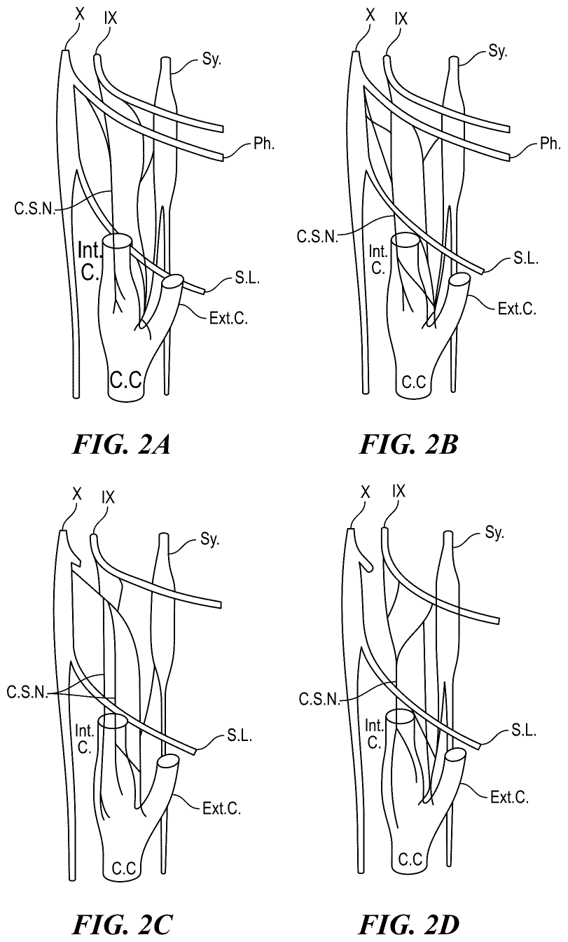

FIGS. 2A-2D are anatomical illustrations showing different patterns of the vagus nerve in the area of the carotid sinus. Referring to FIGS. 2A-2D together, in addition to the CSN, the inter-carotid plexus contains afferent branches of the vagus nerve X, which are specific to the baroreflex. Four distinct patterns, illustrated in FIGS. 2A-2D, have been identified, and all contain branches of the vagus nerve X in the inter-carotid plexus.

The CSN and the vagus nerve X both include afferent nerve fibers, which carry signals to the central nervous system, and efferent nerve fibers, which carry signals away from the central nervous system to peripheral effectors. In some embodiments, the systems, devices, and methods described herein involve stimulating carotid sinus afferent nerve fibers and cardiac-specific vagal afferent nerve fibers, in order to treat hypertension and/or other suitable conditions. In some embodiments, one or both of these types of nerve fibers (i.e., carotid sinus afferent nerve fibers and/or cardiac-specific vagal afferent nerve fibers) may be identified before they are stimulated. For the purposes of this disclosure, carotid sinus afferent nerve fibers may be generally referred to as “the carotid sinus nerve,” “CSN,” and cardiac-specific vagal afferent nerve fibers and/or other vagal fibers that reside within or proximate to the carotid sinus (and/or the bifurcation thereof) may be generally referred to as “the vagus nerve.” In some embodiments, for example, electrodes of the system described herein may be placed on, over or around the CSN and the vagus nerve, and such electrodes may be used to stimulate carotid sinus afferent nerve fibers and/or cardiac-specific vagal afferent nerve fibers. Nerves and/or nerve fibers innervating one or more baroreceptors (e.g., baroreceptor afferents) and/or otherwise associated with the baroreceptor reflex of a patient may be defined or generally referred to herein as “fibers associated with the baroreceptors.”

### III. Sensing Cardiac Depolarization and/or Stimulating Baroreceptors, and Associated Systems, Devices, and Methods

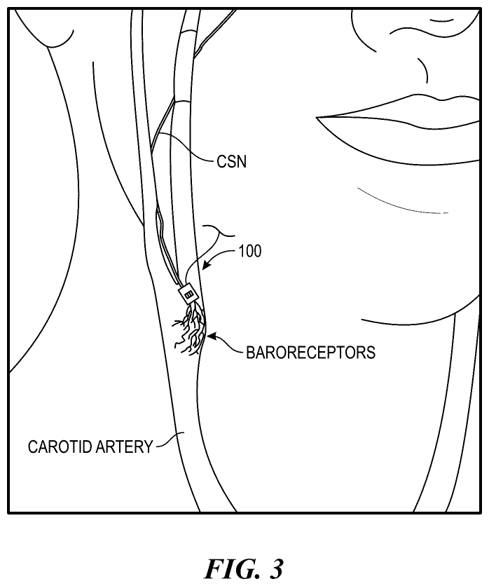

FIG. 3 is a partially schematic illustration of a patient treatment system 100 (“system 100”) implanted at a CSN of a patient, in accordance with embodiments of the present technology. The system 100 can be disposed around the CSN to target some or all of the baroreceptor afferent nerve fibers within the CSN. The system 100 may be delivered subcutaneously to the area of interest and placed over the plexus of nerves that includes the CSN and the vagus nerve, as described above in reference to the anatomical drawings in FIGS. 1A-2D. As described herein, the lead body of the signal delivery device of the system 100 can include a first region having first lead electrodes, and a second region having second electrodes, e.g., that is partially or entirely positionable over the first region. In some embodiments, the lead body can be a flat cuff shape or book shaped, in that the lead body is connected along one edge and open along an opposing edge. The open edge may be turned open to partially envelop or surround one or more nerves, and then closed to hold the nerve(s). As such, the lead electrodes can span across the lead body along a first axis extending in a first direction, and the nerve(s) can extend along a second direction angled and/or normal to the first direction. In some embodiments, the lead and/or base electrodes described herein envelop or abut the mobilized tissue (e.g., are positioned to directly contact the tissue), and can be affixed in one or more alternative manners compared to the embodiment described above. For example, the lead and/or the base electrodes can be positioned in one or more arrangements, including but not limited to a spiral cuff arrangement, a flat cuff or “book” arrangement, a single surface array/electrode arrangement, etc., either in place of or in combination with any of the electrode arrangements described herein.

As described herein, the system 100 can sense cardiac depolarization (e.g., via electrical or acoustic signals produced therefrom), and modulate therapy based at least in part on the signals. The signals corresponding to the cardiac depolarization can be sensed via vectors formed from various combinations of the base electrodes and/or the lead electrodes of the signal delivery device, as well as other input/output devices (e.g., accelerometers or other acoustic devices) of the system 100. Additionally, sensing of the signals can be used to map the tissue of the patient, and therein aid to position the lead body of the signal delivery device in a desired location (e.g., proximate the CSN afferent fibers of the patient).

Additionally or alternatively, the tissue of the patient can be mapped by imaging the tissue, for example, using micro-Optical Coherence Tomography (OCT) imaging, near-infrared fluorescence imaging, Magnetic Resonance (MR) cranial nerve imaging, ultrasound, and/or other suitable imaging techniques. The target neural fibers of the patient can be identified in the image(s) and used to aid in positioning the lead body at the desired location. In these and/or other embodiments, one or more patient tissues can be mapped by applying a stimulus (e.g., an electrical stimulus, via a temporary or chronic stimulator) and observing the patient's response (e.g., motor response, hemodynamic response, etc.) to the applied stimulus. The system 100 can determine one or more physiological parameters of the patient and, once the lead body is in the desired position, modulate therapy to be delivered to the patient based at least in part on the physiological parameters. Therapy delivery to the patient, or more particularly to the CSN afferent fibers, can be provided via one or more of the lead electrodes. It is worth noting that although the system 100 primarily includes descriptions related to lead electrodes in one arrangement, the electrodes of system 100 and any of the embodiments herein can include various electrode arrangement types, including, for example, a spiral cuff arrangement, a flat cuff or “book” arrangement, or a single surface array/electrode arrangement, either in place of or in combination with any of the electrode arrangements described herein.

Additionally or alternatively, the system 100 can include one or more sensors configured to detect muscle fasciculation and the therapy can be modulated based, at least in part, on one or more muscle fasciculations detected by the one or more sensors.

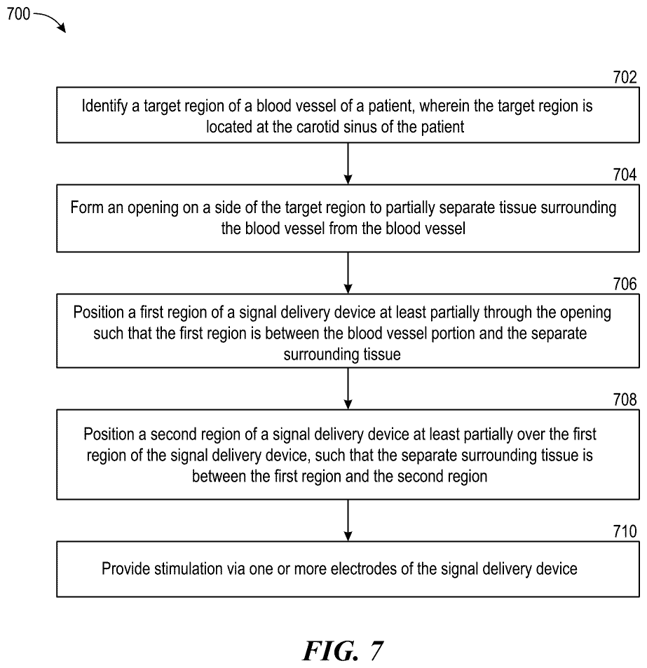

In some embodiments, the system 100 is positioned based on feedback from an electrical stimulus applied via one or more of the electrodes of the system 100. For example, the electrodes of the system 100 can be positioned for implantation based on one or more anatomical landmarks, and electrical stimulation can be applied to verify positioning based on a desired hemodynamic response. If there is not a desired response, the electrode can be repositioned and the process can be repeated, as described in more detail with reference to FIG. 7. In some embodiments, a temporary electrode (e.g., a lead electrode in various arrangements) that is not permanently coupled to the system 100 is used to position the system 100. The temporary electrode can be positioned and repositioned multiple times at the target region before and/or after physical dissection of the tissue at the target region. For example, the temporary electrode can be used after exposure of the carotid bifurcation but prior to dissection of any flap of tissue to incorporate within a permanently implanted electrode. Additionally or alternatively, the temporary electrode can be used on the skin of the patient to identify the positioning of the CSN by applying electrical stimulation to the skin and monitoring for hemodynamic or other responses, such as motor or electrical sensing responses, before dissection.

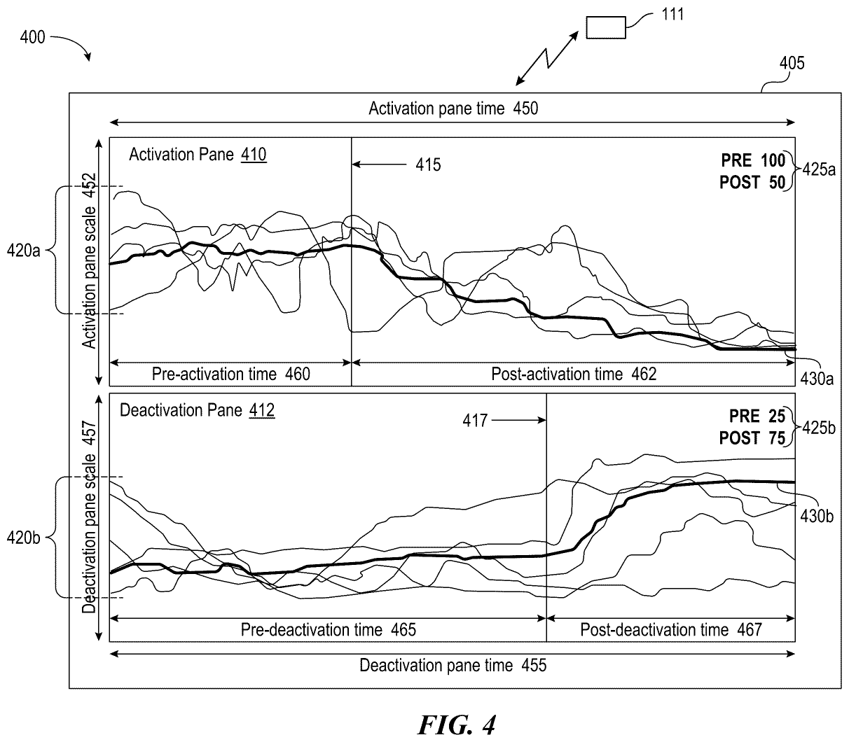

Additionally or alternatively, sensors or sensing devices/systems external (i.e., electrically decoupled) to the system 100 can aid in positioning the system 100. These sensors or sensing devices/systems can include one or more of the modalities described above and can further include other sensors/devices/systems capable of stimulating and/or logging a patient's response to stimuli or changes in physiological parameters. Examples of such modalities include pulse oximeters, electroencephalogram (EEG) electrodes, nerve locator devices, or similar equipment. The parameters measured can include pulse oximeter readings, waveform measurements, waveform-derived parameters, afferent CSN activity, glossopharyngeal nerve activity, other surrounding nerve traffic, EEG recordings, and/or the like. For example, the system 100 can be coupled to or used with a probe configured to take one or more of the measurements described above. In some embodiments, the sensors are sensors readily available in an operating room, such as electrocardiogram (ECG) and intra-arterial pressure sensors. Additionally or alternatively, it can be valuable to use a neuromonitoring system capable of providing generally more detailed and comprehensive monitoring, such as in scenarios of deep anesthesia, generally difficult anatomy, or cases where the baroreflex fibers have been damaged. An example neuromonitoring system is described in more detail with reference to FIG. 4.

FIG. 4 is a partially schematic illustration of a carotid sinus nerve stimulation (CSNS) neuromonitoring system 400, in accordance with embodiments of the present technology. The CSNS neuromonitoring system 400 can include a display 405 that displays one or more panes dedicated to monitoring various physiological signals or time periods in relation to the stimulus provided to a patient. For example, as shown in FIG. 4, the CSNS neuromonitoring system 400 can include an activation pane 410 and a deactivation pane 412 (collectively referred to as “panes 410, 412”). The activation pane 410 can include a synchronization point aligned with the initiation of stimulation to a patient (i.e., from the system 100 or a nerve stimulation probe). Similarly, the deactivation pane 412 can include a synchronization point aligned with the termination of stimulation to a patient (i.e., from the system 100 or a nerve stimulation probe). The CSNS neuromonitoring system 400 can display one or more signal sweeps 420a-b (collectively referred to as “signal sweeps 420”) used to visualize how one or more physiological parameters of the patient change in response to stimuli over a period. The activation pane 410 can include the signal sweep 420a corresponding to the changes in physiological parameters during an activation period. The deactivation pane 412 can include the signal sweep 420b corresponding to the changes in physiological parameters during a deactivation period.

In some embodiments, the CSNS neuromonitoring system 400 is used to position the system 100 by monitoring a patient's response to various stimuli, for example, feedback from electrical stimuli made by the electrodes of the system 100 or stimuli from an external device such as a nerve stimulation probe. The CSNS neuromonitoring system 400 can be operatively coupled to an external device 111 (e.g., one or more controllers, physician's programmers, etc.) able to control and carry out neuromonitoring done by the CSNS neuromonitoring system 400. In some embodiments, the external device 111 receives, processes, adjusts, and/or displays physiological parameters received by the CSNS neuromonitoring system 400. Additionally or alternatively, the external device 111 can be operably coupled to other subsystems (e.g., additional external devices, a power source, etc.) to provide and/or enhance functionality of the CSNS neuromonitoring system 400.

In some embodiments, the external device 111 includes a transmitter and/or receiver enabling the external device 111 to communicate (e.g., wirelessly communicate) with a remote user interface (e.g., on a mobile device and/or remote network). The external device 111 can be configured to operate the CSNS neuromonitoring system 400 in an active monitoring mode and/or process and provide summary statistics that provide context of the position of the one or more neurological structures. Additionally or alternatively, the external device 111 can be configured to receive physiological data from the system 100 and/or external sensors, systems, or devices and control the display 405 based on the received inputs. For example, the external device 111 can be used to filter and calibrate one or more physiological signals into the signal sweeps 420 to be displayed on the display 405, providing a more concise view of the patient's response to stimuli and thus the position of the surrounding neurological structures. Additionally or alternatively, the external device 111 can utilize artificial intelligence or machine learning to adjust signals or other control parameters, e.g., based on previous parameters used for similar patients. It is noted that the external device 111 of FIG. 4 is omitted from some of the other figures but can be included in all embodiments disclosed herein.

The signal sweeps 420 can refer to a graphical representation of physiological data over a specific period of time. The physiological signals can be derived directly from the system 100 and/or from one or more sensors, systems, or devices external to the system 100, such as an implanted pulse generator (IPG) or an external pulse generator (EPG). Although the signals described herein are related to the signal sweeps 420, the signals displayed in one or both of the panes 410, 412 can be raw data signals over time, such as a BP waveform. Additionally or alternatively, the signals can be pre-processed, for example, by the external device 111 into tachograms derived from an ECG. The external device 111 can include an underlying signal acquisition system with capabilities for signal amplification, filtering, calibration, etc.

The signal sweeps 420 displayed in the panes 410, 412 do not necessarily scroll continuously with time. Rather, the signal sweeps 420 can represent a consolidated and automatically aligned version of the physiological parameters across a fixed period of time. The signal sweeps 420 can be calculated retrospectively by the CSNS neuromonitoring system 400 (e.g., by the external device 111) or by a system external to the CSNS neuromonitoring system 400. The signal sweeps 420 can provide a clear and concise view of the patient's physiological responses and thus a method for positioning by monitoring and analyzing neural activity (e.g., neural activity of the CSN and surrounding neural structures). The panes 410, 412 can be aligned with stimulation by detecting the presence or absence of stimulation through direct detection and/or a flag provided by the stimulator and/or the physician (e.g., through interaction with the system 100, the display 405, and/or the external device 111). The panes 410, 412 can be independent of each other and configured with unique settings.

Additionally or alternatively, the external device 111 can automatically label one or more axis and/or components of the signal sweeps 420 corresponding the panes 410, 412. For example, the activation pane 410 can include an activation pane time axis 450 (i.e., corresponding to an X-axis) and an activation pane scale axis 452 (i.e., corresponding to a Y-axis). The activation pane scale axis 452 can change depending on the physiological signals being displayed in the activation pane 410. Additionally or alternatively, the activation pane 410 can include a line 415 representing a time of activation that demarcates a pre-activation time period 460 and a post-activation time period 462. In some embodiments, the deactivation pane 412 includes a deactivation pane time axis 455 (i.e., corresponding to an X-axis) and a deactivation pane scale axis 457 (i.e., corresponding to a Y-axis). Similar to the activation pane scale axis 452, the deactivation pane scale axis 457 can change depending on the physiological signals being displayed in the deactivation pane 412. Additionally or alternatively, the deactivation pane 412 can include a line 417 representing a time of deactivation that demarcates a pre-deactivation time period 465 and a post-deactivation time period 467.

Additionally or alternatively, the panes 410, 412 can include generally similar labels that make comparison of the signal sweeps generally easier for a user (e.g., a physician) to visualize and interpret. For example, the panes 410, 412 can include summary statistics 425a-b (collectively referred to as “summary statistics 425”) and composite signals 430a-b (collectively referred to as “composite signals 430”). In some embodiments, the summary statistics 425 display the patient's heart rate, blood pressure, and/or other physiological parameters affected by the electrical stimulation provided to the patient. Additionally or alternatively, the composite signals 430 can include the physiological signal displays after it has averaged and/or adjusted using one or more calibration techniques, for example, for noise. The composition of the composite signal can be controlled by the user such that the same or different averaging and/or adjusting processes are done for certain physiological signals, for certain signals received from certain device, and/or the like.

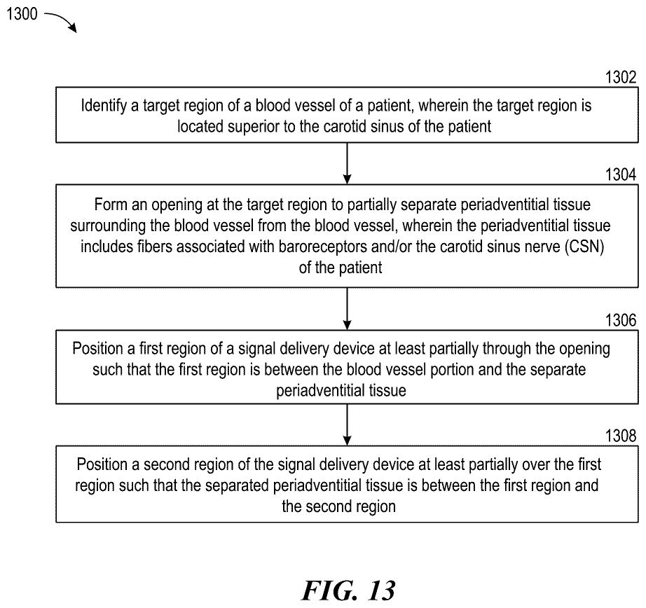

In some embodiments, the user reviews the display from one or both signals shown in the activation pane 410 and/or the deactivation pane 412 to draw conclusions regarding the positioning of neurological structures relative to where electrical stimulation was applied. The CSNS neuromonitoring system 400 can analyze the patient's response to electrical stimulation, and/or compare the displayed signals with known response patterns, which can be used to verify the positioning of the signal delivery device relative to neurological structures by ensuring that the observed responses align with expected outcomes for targeted neurological structures. Additionally or alternatively, the CSNS neuromonitoring system 400 can assess whether the patient's response is less than, within range, or greater than a threshold response by monitoring physiological parameters such as blood pressure and heart rate, providing insights into the effectiveness of the stimulation and thus the accuracy of the device placement, as described in more detail with reference to FIGS. 7 and 13.

In some embodiments, one pane with one sweep is sufficient to detect the positioning of the neurological structures, but in challenging cases, multiple panes with many signal sweeps can be calibrated by the CSNS neuromonitoring system 400 and/or displayed on the display 405. One or more of the panes 410, 412 can be cleared by the user to rerun the display process. Additionally or alternatively, the user can make adjustments to controls that are reflected in real time and can be applied retroactively, such as adding past sweeps to the display.

As described above, one or more clinical conclusions can be drawn from raw patient data using the CSNS neuromonitoring system 400. In some embodiments, the clinical conclusions are drawn from raw intra-arterial blood pressure data observed with the CSNS neuromonitoring system 400. For instance, during the activation phase, the clinician can observe a decrease in blood pressure in the post-activation time period 462 compared to the pre-activation time period 460 in the activation pane 410 at the time of deactivation 415. The decrease in blood pressure is not necessarily instantaneous with stimulation but rather exhibited as a downward trend that eventually stabilizes, provided the stimulation is applied for a sufficient duration and the observation window is adequately long. The decrease in signal may not be apparent in individual sweeps, necessitating the clinician to refer to composite sweeps to confirm the presence of the trend. Similarly, in the deactivation phase, the process mirrors that of the activation phase and is observed in the deactivation pane 412. During the pre-deactivation time period 465, the signal (e.g., blood pressure) can be in a steady state. Additionally or alternatively, the signal in the pre-activation time period 465 can be not in a steady state (e.g., the signal could be drifting downward). The clinician can look for a reversal of the signal trend around the time of deactivation 417 (e.g., the signal begins to trend upwards). The same considerations regarding the speed of the trend reversal and the number of sweeps required apply. Furthermore, in the activation phase, if the signal is not in a steady state prior to activation (e.g., drifting upward from a previous deactivation), the clinician can look for an additional trend reversal. The clinical interpretation of these observations can depend on several factors, including the magnitude of the signal change (e.g., the magnitude of blood pressure change), the duration allowed for the pressure to equilibrate (e.g., with longer durations favoring larger changes), the intensity of the stimulation, and/or the like. A larger change in signal over a shorter period of time with lower stimulation intensity provided to the patient is generally viewed more favorably.

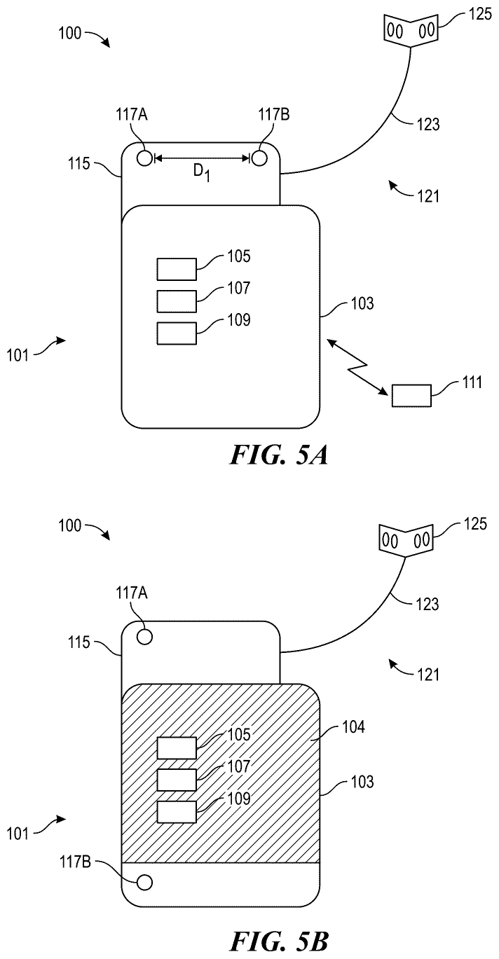

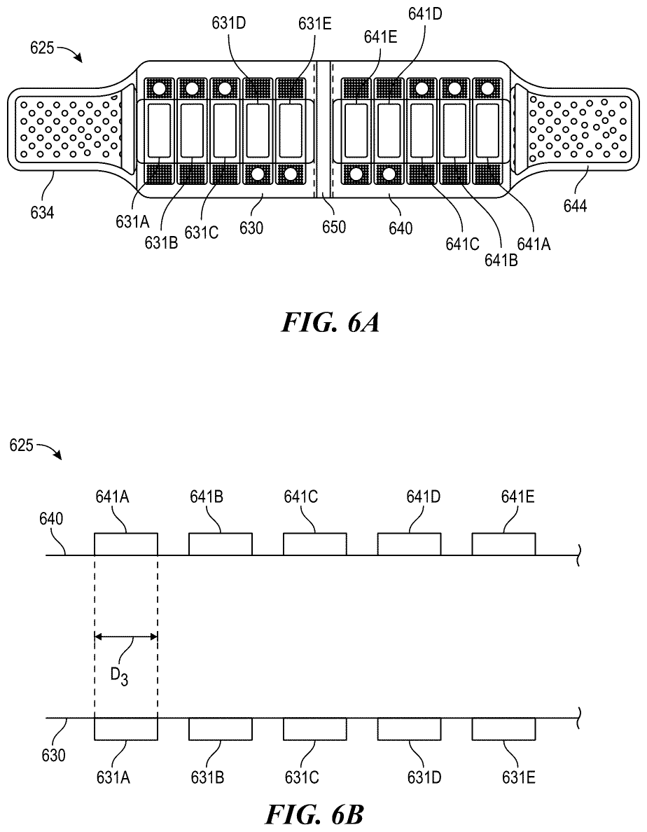

FIGS. 5A and 5B are partially schematic illustrations of various embodiments of the system 100 shown in FIG. 3, in accordance with embodiments of the present technology. Referring to FIGS. 5A and 5B together, the system 100 includes an implantable neuromodulator 101 (e.g., a signal generator or IPG and one or more signal delivery elements or devices 121 (“signal delivery device 121”) electrically coupleable to the neuromodulator 101. The signal delivery device 121 can be implanted within a patient and carry features for delivering therapy to the patient after implantation. The neuromodulator 101 can be connected directly to the signal delivery device 121, or it can be connected to the signal delivery device 121 via a signal link or lead extension. As explained herein (e.g., with reference to FIGS. 6A and 6B), the signal delivery device 121 can include a lead body 125 having one or more lead electrodes, and one or more conductors 123 extending from and electrically coupling the lead electrodes to the neuromodulator 101. As used herein, the terms signal delivery device, lead, and/or lead body include any of a number of suitable substrates and/or support members that carry electrodes/devices for providing therapy signals to a patient. For example, the lead body 125 can include one or more electrodes or electrical contacts that direct electrical signals into the patient's tissue or fibers (e.g., to treat hypertension). In other embodiments, the signal delivery device 121 can include structures other than a lead body that also direct electrical signals and/or other types of signals to the patient.

The neuromodulator 101 can include a housing 103 made from a conductive material (e.g., titanium or other metal), and one or more base electrodes (e.g., contacts) 117A, 117B carried by the housing 103 and spaced apart from one another (e.g., to create a sufficient vector). The base electrodes 117A, 117B can serve as an anode/cathode pair, and can each be electrically coupled to each of the lead electrodes of the lead body 125 via the conductors 123. In some embodiments, the base electrodes 117A, 117B are contacts that are an exposed conductive portion of the housing 103. As shown in FIG. 5A, the base electrodes 117A, 117B are included on a header portion 115 of the neuromodulator 101 and spaced apart from one another by a minimum distance (D₁), which can be at least 1.0 inch, 1.5 inches, or 2 inches. As shown in FIG. 5B, the neuromodulator 101 can include an insulative or non-conductive material 104. In such embodiments, the base electrodes 117A, 117B can be spaced apart from one another along a height of the neuromodulator 101, for example, with one of the base electrodes 117A on the header portion 115 and the other of the base electrodes 117B on another portion of the neuromodulator not covered by the insulative material 104. In some embodiments, the base electrodes of system 100 are positioned on the header portion 115 or as a distant reference electrode in various arrangements, for example, in one or more of the arrangements described herein.

Referring again to FIGS. 5A and 5B together, the neuromodulator 101 can transmit signals (e.g., electrical signals, neuromodulation pulses, etc.) to the signal delivery device 121 that up-regulate or activate (e.g., excite or depolarize one or more axons) and/or down-regulate or inhibit (e.g., block action potential propagation or suppress action potential formation) target nerves. As used herein, and unless otherwise noted, the terms “modulate,” “modulation,” “stimulate,” and “stimulation” refer generally to signals that have either type of the foregoing effects on the target nerves. The neuromodulator 101 can include a machine-readable (e.g., computer-readable) medium containing instructions for generating and transmitting suitable therapy signals. The neuromodulator 101 and/or other elements of the system 100 can include one or more processor(s) 105, memory unit(s) 107, and/or input/output device(s) 109 (“I/O devices 109”). Accordingly, the process of providing modulation signals, providing guidance information for positioning the signal delivery device 121 (e.g., relative to target fibers of the patient), and/or executing other associated functions can be performed by computer-executable instructions contained by, on, or in computer-readable media located at the neuromodulator 101 and/or other system components. The neuromodulator 101 and/or other system components may include dedicated hardware, firmware, and/or software for executing computer-executable instructions that, when executed, perform any one or more methods, processes, and/or sub-processes described herein (e.g., the methods, processes, and/or sub-processes described herein). Said dedicated hardware, firmware, and/or software also serve as “means for” performing the methods, processes, and/or sub-processes described herein. The neuromodulator 101 can also include multiple portions, elements, and/or subsystems (e.g., for directing signals in accordance with multiple signal delivery parameters), carried in a single housing, as shown in FIGS. 5A and 5B, or in multiple housings. In some embodiments, the system 100 includes the external device 111 described in more detail with reference to the CSNS neuromonitoring system 400 of FIG. 4. The external device 111 can be, for example, one or more controllers or physician's programmers able to control and carry out therapy provided via the system 100. In some embodiments, the external device 111 able to control or carry out therapy provided by the system 100 is the same external device 111 able to control and carry out neuromonitoring done by the CSNS neuromonitoring system 400 of FIG. 4. Additionally or alternatively, the external device 111 able to control or carry out therapy provided by the system 100 can be distinct from the external device 111 able to control and carry out neuromonitoring done by the CSNS neuromonitoring system 400 of FIG. 4.

The neuromodulator 101 can also receive and respond to an input signal received from one or more sources. The input signals can direct or influence the manner in which the therapy and/or process instructions are selected, executed, updated, and/or otherwise performed. The input signals can be received from one or more sensors (e.g., the I/O devices 109) that are carried by the neuromodulator 101 and/or distributed outside the neuromodulator 101 (e.g., at other patient locations) while still communicating with the neuromodulator 101. The sensors and/or other I/O devices 109 can provide inputs that depend on or reflect patient state (e.g., patient position, patient posture, patient heart rate, patient blood pressure, patient respiratory rate, patient minute ventilation, and/or patient activity level), and/or inputs that are patient-independent (e.g., time).

In some embodiments, the I/O devices 109 include an accelerometer (e.g., a multi-axial accelerometer or tri-axial accelerometer). In such embodiments, the accelerometer can be used to sense or obtain acoustic signals generated from and/or associated with cardiac depolarization. The acoustic signals can be utilized additionally or alternatively to the electrical signals generated from and/or associated with cardiac depolarization that are sensed at least via the base electrodes 117A, 117B of the neuromodulator 101 and/or the lead electrodes of the lead body 125. Additionally, or alternatively to determining the acoustic signals generated from and/or associated with cardiac depolarization, the accelerometer can be configured to detect acoustic signals associated with airflow, for example, to measure the patient's respiratory rate and/or other respiratory related information (e.g., detecting apneas, hypopneas, snoring, etc.). In these and/or other embodiments, the accelerometer can be used to determine patient position and/or orientation relative to a gravitational field, including whether the patient is standing, sitting, lying down (e.g., sleeping), etc. In such embodiments, the accelerometer can serve, for example, as a fall detector or safety mechanism, and the signal from the accelerometer can be used to adjust stimulation or characteristics of the neuromodulation pulses. In some embodiments, the I/O devices 109 include a tonometer for determining arterial stiffness, or other devices for determining an augmentation pressure waveform or index. As described herein, arterial stiffness and/or the augmentation pressure waveform or index can be used as a physiological parameter that in part affects the characteristics of the neuromodulation pulses provided to the patient via the signal delivery device 121.

In some embodiments, data from the accelerometer is used to detect whether the patient is asleep and/or the patient's actual or expected sleep state (e.g., REM, non-REM, etc.). For example, changes (or a lack thereof) to the physical orientation and/or movement of the accelerometer can indicate when the patient has been supine or otherwise immobile for extended periods of time which, in turn, can indicate that the patient is asleep. In some embodiments, the data from the accelerometer (e.g., one or more acoustic signals, patient position, patient orientation, etc.) is compared with data from one or more other sensors (e.g., heart rate sensors) to detect whether the patient is asleep and/or the patient's actual or expected sleep state. These and/or other data associated with whether the patient is asleep and/or the patient's sleep states can be used to adjust the neuromodulation pulses delivered to the patient. For example, an intensity of the neuromodulation pulses can be reduced during non-REM sleep to conserve energy, for example, because the patient's sympathetic nervous system activity is expected to be lower or at a minimum during these times.

In some embodiments, the I/O devices 109 are configured to detect and/or receive an input from the patient corresponding to an activity or state of the patient. For example, the I/O devices 109 can include a software application configured to allow the user to select one or more profiles associated with the patient's physical, mental, and/or emotional state. These can include, for example, participating in structured exercise (e.g., cardio, such as jogging, elliptical, walking, biking, swimming, etc.; strength training, such as weightlifting; isometric exercise, such as yoga; sit-ups; push-ups; etc.), inactive wakefulness, sleep, anxious or stressed, meal-time and/or post-prandial, bearing down (e.g., bowel movement), intercourse, etc. These and/or other physiological parameters can be used to adjust the neuromodulation pulses delivered to the patient. For example, respiratory sensing as described above and herein can include measuring a patient's respiration using various techniques, including but not limited to impedance and accelerometer methods, for the purposes of activity monitoring and/or monitoring breathing during sleep. In some embodiments, it is advantageous to monitor a patient's respiration during sleep for both diagnostic and therapeutic functions, as baroreflex fiber stimulation has the potential to influence airway patency, addressing obstructive sleep apnea, as well as spontaneous respiratory activity, addressing central sleep apnea.

In some embodiments, the neuromodulator 101 and/or signal delivery device 121 obtain power to generate the therapy signals from an external power source (not shown). In some embodiments, the external power source transmits power to the implanted neuromodulator 101 and/or directly to the signal delivery device 121 using electromagnetic induction (e.g., RF signals). For example, the external power source can include an external coil that communicates with a corresponding internal coil within the implantable neuromodulator 101, signal delivery device 121, and/or a power relay component. The external power source can be portable for ease of use.

FIGS. 6A and 6B are partially schematic illustrations of a lead body 625 of a signal delivery device 621 configured in accordance with embodiments of the present technology. FIG. 6A illustrates a plane view of the lead body 625 in an open configuration and FIG. 6B illustrates a cross-sectional view of the lead body 625 in a closed configuration. The signal delivery device 621 shown and described with reference to FIGS. 6A and 6B can include any one or more of the features of and/or be generally similar to the signal delivery device 121 of FIGS. 5A and/or 5B. Additionally, the signal delivery device 121 can include any one or more of the features of one or more of the other signal delivery devices described herein.

As shown in FIG. 6A, the signal delivery device 621 includes a lead body 625 including a first region 630 (e.g., a first plate, first face, first substrate, etc.), a second region 640 (e.g., a second plate, second face, second substrate, etc.), an intermediate region 650 between the first region 630 and the second region 640, and one or more lead electrodes 631, 641. The first region 630 can include a first end portion 634 opposite the intermediate region 650 and/or the second region 640 can include a second end portion 644 opposite the intermediate region 650. One or both of the end portions 634, 644 can include grip tabs, rounded edges, and/or other features to facilitate implantation. In at least some embodiments, for examples, the end portions 634, 644 can include suture holes and/or otherwise be configured to be sutured or coupled to one another, e.g., after implantation. For example, one or more surgical clips (e.g., vessel clips) can contact or engage the end portions 634, 644 to couple the end portions 634, 644 to one another.

The lead electrodes 631, 641 can be positioned on one or more sides (e.g., a front side, a back side, etc.) of the lead body 625, and can include a first set of lead electrodes 631A-631E (collectively referred to as “the first lead electrodes 631”) on the first region 630, and a second set of lead electrodes 641A-641E (collectively referred to as “the second lead electrodes 641”) on the second region 640. The first lead electrodes 631 can be aligned on the first region 630 in a direction at least generally perpendicular to the intermediate region 650 (e.g., as shown in FIG. 6A) or in a direction at least generally parallel to the intermediate region 650. Similarly, the second lead electrodes 641 can be aligned on the second region 640 in a direction at least generally perpendicular to the intermediate region 650 (e.g., as shown in FIG. 6A) or in a direction at least generally parallel to the intermediate region 650. Returning to FIG. 6A, individual ones of the first lead electrodes 631 and/or the second lead electrodes 641 can have a length and/or a width of up to 5 mm, such as up to 4 mm, up to 3 mm, up to 2 mm, up to 1 mm, up to 0.5 mm, etc. In at least some embodiments, for example, one or more of the first lead electrodes 631 and/or one or more of the second lead electrodes 641 have a length of 2 mm and a width of 0.8 mm. Although the first and second lead electrodes 631, 641 have a rectangular shape in the embodiment illustrated in FIG. 6A, in other embodiments, individual ones of the first and/or second lead electrodes 631, 641 can have a circular, oval, square, pentagonal, hexagonal, ring, “X,” zig-zag, and/or other suitable shape. Each of the first lead electrodes 631 and the second lead electrodes 641 can be a positively charged electrode or a negatively charged electrode. In some embodiments, the first lead electrodes 631 and the second lead electrodes 641 include alternatively charged electrodes. For example, the primary first lead electrode 631A can be positively charged, the secondary first lead electrode 631B can be negatively charged, the tertiary first lead electrode 631C can be positively charged, and so on and so forth. In some embodiments, all the first lead electrodes 631 are positively charged electrodes and all of the second lead electrodes 641 are negatively charged electrodes (or vice versa). The first lead electrodes 631 and the second lead electrodes 641 can each span the same distance (D₂) of the respective first region 630 and second region 640. The first region 630 can be positioned over the second region 640, for example, by folding the first region 530 over the second region 640 (or vice versa) about the intermediate region 650. Individual pairs of the first and/or second lead electrodes 631, 641 can be referenced against one or more other pairs of the first and/or second lead electrodes 631, 641 (e.g., to determine a relative impedance, therapy delivery efficacy, to map the patient's tissue, etc.).

As shown in FIG. 6B, the second region 640 is positioned over the first region 630 and, in such a configuration, individual first lead electrodes 631 are positioned over or at least partially over (e.g., at least partially aligned with and/or overlapping) corresponding individual second lead electrodes 641. For example, when the first region 630 is positioned over the second region 640, the primary first lead electrode 631A is positioned over the primary second lead electrode 641A, the secondary first lead electrode 631B is positioned over the secondary second lead electrode 641B, and so on and so forth. In some embodiments, the individual first lead electrodes 631 and the individual second lead electrodes 641 span the same distance (D₃) of the corresponding first region 630 or second region 640 and/or overlap completely with one another. In other embodiments, one or more of the individual first lead electrodes 631 can span a different distance of the corresponding first region 630 or second region 640 and/or be offset relative to one another.

In operation, the signal delivery device 621 (and, more particularly, the lead body 625) can be positioned around a target area or nerve(s) (e.g., afferent nerve fibers) including the CSN. For example, the first region 630 and/or first lead electrodes 631 can be on a first side of the target nerve(s) and the second region and/or second lead electrodes 641 can be on a second, opposing side of the target nerve(s). The signal delivery device 621 can deliver neuromodulation pulses to the target nerve(s) via one or more of the lead electrodes, for example, as monopolar stimulation or multi-polar stimulation (e.g., bipolar stimulation, tripolar stimulation, etc.). For example, neuromodulation pulses can be delivered as monopolar stimulation via one of the first lead electrodes 631 or one of the second lead electrodes 641, or as bipolar stimulation via a combination of the first and second lead electrodes 631, 641 (e.g., the primary first lead electrode 631A and the primary second lead electrode 641A). Additionally, or alternatively, the first lead electrodes 631 and/or second lead electrodes 641 can be positively biased (+) or negatively biased (−) and have a number of arrangements. For example, adjacent electrodes can have arrangements including +−−+, +−+, −++−, −+−, +−−−+, −+++−, or +−, amongst other possibilities, and opposing electrodes (e.g., when the second region 640 is positioned over the first region 630) can be complementary biased. Advantageously, the arrangement of the first lead electrodes 631 relative to the second lead electrodes 641 can decrease the energy needed to deliver stimulation to the target nerve. Stated differently, by arranging individual lead electrodes on the first region 630 and the second region 640 that are opposed to one another and on opposing sides of the target nerve, embodiments of the present technology can enable bipolar stimulation to be delivered that target particular nerves, while also minimizing the amount of energy required to do so.

As shown in FIGS. 6A and 6B, the signal delivery device 621 includes ten lead electrodes. However, in other embodiments, the signal delivery device 621 can include more or fewer (e.g., two, three, four, five, six, seven, eight, nine, twelve, fourteen, fifteen, sixteen, or twenty) lead electrodes. Additionally, or alternatively, the lead electrodes may be arranged in a different configuration, including any of the configured described in U.S. patent application Ser. No. 18/333,416, the entirety of which was previously incorporated by reference and is reproduced in Appendix A. It is also worth noting that the lead electrodes described above and herein (e.g., the first lead electrodes 631 and/or the second lead electrodes 641) can additionally or alternatively be arranged in various configurations, such as a spiral cuff arrangement or a flat cuff or “book” arrangement. The signal delivery device 621 can be configured to accommodate a plurality of such electrode arrangements.

FIG. 7 is a flow diagram of a method 700 for positioning a patient treatment system, in accordance with embodiments of the present technology. The method 700 is illustrated as a series of steps, process portions, or blocks 702-710. At block 702, the method 700 can include identifying a target region of a blood vessel of a patient. The target region can include and/or extend around all, or at least a portion, of a circumference of the blood vessel and/or can be located at, or at least proximate to (i) the carotid arteries including the CCA, ICA, and/or ECA, (ii) the carotid sinus, (iii) the CSN, (iv) the glossopharyngeal nerve (i.e., cranial nerve IX), (v) the ascending pharyngeal artery, (vi) the superior thyroid artery, (vii) the hypoglossal nerve (i.e., cranial nerve XII), (viii) the vagus nerve (i.e., cranial nerve X), (ix) the carotid body, and/or (x) the superior cervical ganglion. In some embodiments, the blood vessel includes the CCA and/or the ICA and identifying the target region includes identifying a target region of the CCA and/or the ICA. A portion of the CSN and/or one or more fibers associated with the baroreceptors runs along a length of the ICA and can pass through the target region. Accordingly, by virtue of its location relative to other portions of the carotid sinus, the target region is expected to include the CSN and/or one or more fibers associated with the baroreceptors. In some embodiments, identifying the target region includes identifying a portion of a circumference of the ICA expected to include the CSN and/or one or more fibers associated with the baroreceptors. In these and/or other embodiments, identifying the target region can include using ultrasound, OCT, and/or other imaging techniques to identify the target region and/or confirm that the CSN and/or one or more fibers associated with the baroreceptors are present in the target region.

At block 704, the method 700 can include forming an opening on a side of the target region. For example, the target region can include a first side and a second side opposite the first side. Forming the opening can include forming an opening in the first side and/or in the second side. When formed, the opening can extend at least partially or fully between the first side and the second side, e.g., with a first end of the opening formed at the first side and/or a second end of the opening formed at the second side.

A blood vessel (e.g., artery or vein) can include intimal tissues (e.g., endothelium, basement membrane, stratum subendothelial, internal clastic membrane), medial tissues (e.g., smooth muscle, collagen), and adventitial tissues (e.g., external elastic membrane, serosa, nerves, blood vessels). These tissues can be surrounded by periadventitial and/or other outermost connecting tissues. In some embodiments, forming the opening referred to herein includes forming the opening to partially separate periadventitial tissue from the underlying tissues of the blood vessel (e.g., intimal, medial, and adventitial tissues). The periadventitial tissue, positioned on one side of the opening, can include the CSN and/or the fibers associated with the baroreceptors. The opening can separate the periadventitial tissue from the blood vessel, such that the periadventitial tissue remains attached to the blood vessel except at the first side, the second side, and/or the opening extending therebetween.

At block 706, the method 700 can include positioning a first region of a signal delivery device at least partially through the opening (block 704). The signal delivery device can be or can be a part of one or more of the patient treatment systems described herein. Positioning the first region of the signal delivery device at least partially through the opening can include positioning the first region between the separated surrounding (e.g., periadventitial) tissue and the blood vessel. For example, positioning the first region of the signal delivery device at least partially through the opening can include positioning the first region 630 of the signal delivery device 621 (FIG. 6A) through the opening, between the periadventitial tissue and the blood vessel. In such embodiments, the first lead electrodes 631 (FIG. 6A) carried by the first region 630 can face toward the periadventitial tissue, the CSN, and/or the one or more fibers associated with the baroreceptors. In other embodiments, the second region 640 (FIG. 6A) of the signal delivery device 621 can be positioned at least partially through the opening, e.g., with the second lead electrodes 641 (FIG. 6A) carried by the second region 640 facing the periadventitial tissue, the CSN, and/or the one or more fibers associated with the baroreceptors.

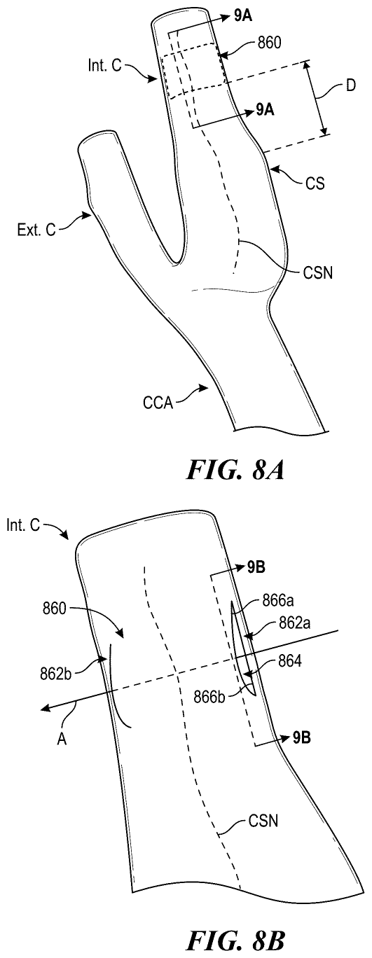

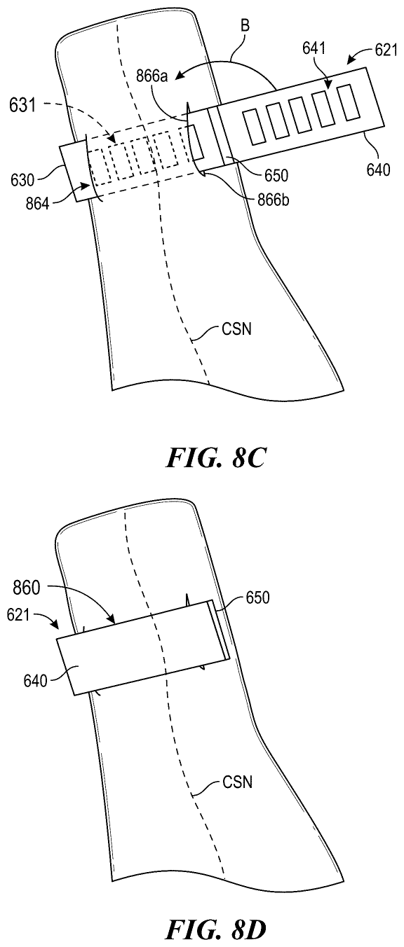

As shown in FIG. 7, at block 708, the method 700 can include positioning a second region of the signal delivery device at least partially over the first region of the signal delivery device. Positioning the second region at least partially over the first region can include positioning the separated surrounding tissue between the first region of the signal delivery device and the second region of the signal delivery device. For example, positioning the second region at least partially over the first region can include positioning the second region 640 (FIGS. 6A and 6B) at least partially over the first region 630 (FIGS. 6A and 6B). In some embodiments, the second region 640 is folded about the intermediate region 650 to position the second region 640 at least partially over the first region 630. With the second region 640 in this position, the target region and, e.g., the CSN and/or the one or more fibers associated with the baroreceptors, can be positioned between the first region 630 and the second region 640. Also in this position, individual electrodes of the first and second lead electrodes 631, 641 carried by the first and second regions 630, 640 can be aligned with one another as shown in FIG. 6B, e.g., with the separated surrounding tissue, the CSN, and/or the one or more fibers associated with the baroreceptors positioned between the first and second lead electrodes 631, 641. For embodiments in which the second region has been positioned within the opening, block 708 can include positioning the first region 630 (FIG. 8C) over the second region 640.

As shown in FIG. 7, at block 710, the method 700 can include providing stimulation (e.g., to the nerves and/or fibers within the separated surrounding tissue) via one or more electrodes of the signal delivery device. Providing stimulation can include delivering an electrical signal to the CSN and/or the one or more fibers associated with the baroreceptors via one or more of the electrodes, such as the electrodes 631, 641 (FIGS. 6A, 6B, 8C), carried by the signal delivery device. In some embodiments, stimulating the CSN afferent fibers includes delivering a test stimulation and observing a patient response to the delivery of the test stimulation, e.g., to confirm that the CSN and/or the one or more fibers associated with the baroreceptors are positioned between the first and second regions of the signal delivery device. Representative stimulation algorithms and methods are described previously herein and/or in U.S. patent application Ser. No. 18/333,416, the disclosure of which is incorporated herein by reference in its entirety and reproduced in Appendix A.

In some embodiments, providing stimulation includes identifying one or more electrodes to deliver the stimulation. This can include, for example, selecting a first electrode of the signal delivery device to deliver a first electrical signal to the patient, delivering the first electrical signal via the first electrode, and determining a patient response to the electrical signal, such as a change in heart rate and/or blood pressure. If the patient response is less than a response threshold, for example, a change in heart rate of less than 5% or 10% and/or a change in blood pressure of less 10 mmHg, then the method 700 can further include delivering a second electrical signal via a second electrode of the signal delivery device different than the first electrode and determining the patient's response to the second electrical signal. This process can be repeated until the patient's response meets or exceeds the response threshold.

Additionally or alternatively, signal delivery can be monitored by observing the stimulation's impact on the baroreflex in various manners. For example, the patient can receive a simple stimulation sequence such as an “OFF-ON-OFF-ON-OFF” sequence while a physician visually analyzes blood pressure changes using a display, monitor, or external device. In some embodiments, a neuromonitoring system is used to monitor signal delivery from the signal delivery device, as described in more detail in FIG. 4.

In some embodiments, providing stimulation includes identifying one or more signal delivery parameters at which the stimulation is to be delivered. This can include, for example, selecting a first value of a signal delivery parameter at which the signal delivery device delivers an electrical signal to the patient, delivering the electrical signal at the first value, and determining a patient response to the electrical signal. The signal delivery parameter can include a frequency, amplitude, pulse width, interpulse time interval, duty cycle, a range or series of signal delivery parameters (e.g., a frequency decay or crescendo, an amplitude ramp, etc.) and/or one or more other suitable signal delivery parameters. The patient response can include a change in blood pressure, as described previously herein. If the patient response is less than the response threshold, the method 600 can further include delivering the electrical signal at a second value of the signal delivery parameter, different than the first value, and determining the patient's response to the electrical signal with the second value. In some embodiments, the response threshold is time-varying. This process can be repeated until the patient's response meets or exceeds the response threshold.

FIGS. 8A-8D are illustrations of the CCA and the signal delivery device 621 of FIGS. 6A and 6B during different stages of the method 700, in accordance with embodiments of the present technology. Referring to FIG. 8A, a practitioner can identify a target region 860 located on the ICA (i.e., Int. C), as described previously with reference to block 702. The target region 860 can extend around all, or at least a portion, of a circumference of the ICA, and/or can be positioned a distance D superior to the carotid sinus. The distance D can be at least 0.5 cm (centimeters), 1 cm, 1.5 cm, 2 cm, another distance therebetween, and/or within a range of 0.5 and 5 cm. In some embodiments, the target zone is adjacent a superior border of the carotid sinus) and thus the distance D is less than 0.5 cm or between 0 cm and 0.5 cm. In some embodiments, the target region 860 varies in location and/or size (e.g., the amount of tissue at or around the target region 860). For example, duc to variations in the micro-neuroanatomy from patient to patient, the target region 860 may not be within the exact distance D stated above. Other methods of target arca identification can be used in addition to or in place of identifying the target region through the landmarks and distance D described above.

Referring to FIG. 8B, the practitioner can form an opening 864 that separates periadventitial tissue 866a (e.g., outer portion) surrounding the ICA from an ICA 866b (e.g., inner portion), as described previously with reference to block 704. To form the opening 864 and/or separate the periadventitial tissue 866a from the underlying ICA 866b, the practitioner can form an incision between the periadventitial tissue 866a and the ICA 866b along a length of the ICA at a first side 862a of the target region 860 and at a second side 862b of the target region 860. In the illustrated embodiment, the opening 864 extends from the first side 862a toward and/or to the second side 862b in the direction indicated by arrow A. The periadventitial tissue 866a can be separated from the ICA 866b by the opening 864, such that the periadventitial tissue 866a remains attached to the ICA 866b except at the first side 862a, the second side 862b, and/or the opening 864 extending therebetween.

Referring to FIG. 8C, the first region 630 of the signal delivery device 621 can be positioned at least partially through the opening 864, e.g., between the periadventitial tissue 866a and the underlying ICA 866b, e.g., as described previously with reference to block 706. The first lead electrodes 631 carried by the first region 630 can face toward the periadventitial tissue 866a, the CSN, and/or the one or more fibers associated with the baroreceptors of the patient. In other embodiments, the second region 640 of the signal delivery device 621 can be positioned at least partially through the opening 864, e.g., with the second lead electrodes 641 carried by the second region 640 facing the periadventitial tissue 866a, the CSN, and/or the one or more fibers associated with the baroreceptors.

Referring to FIG. 8D, the second region 640 can be folded about the intermediate region 550 in the direction indicated by arrow B, as shown in FIG. 8C, to position the second region 640 at least partially over the first region 630, as shown in FIG. 8D. In this position, the second region 640 overlays the target region 860, which can include the CSN and/or the one or more fibers associated with the baroreceptors, as described previously with reference to block 708.

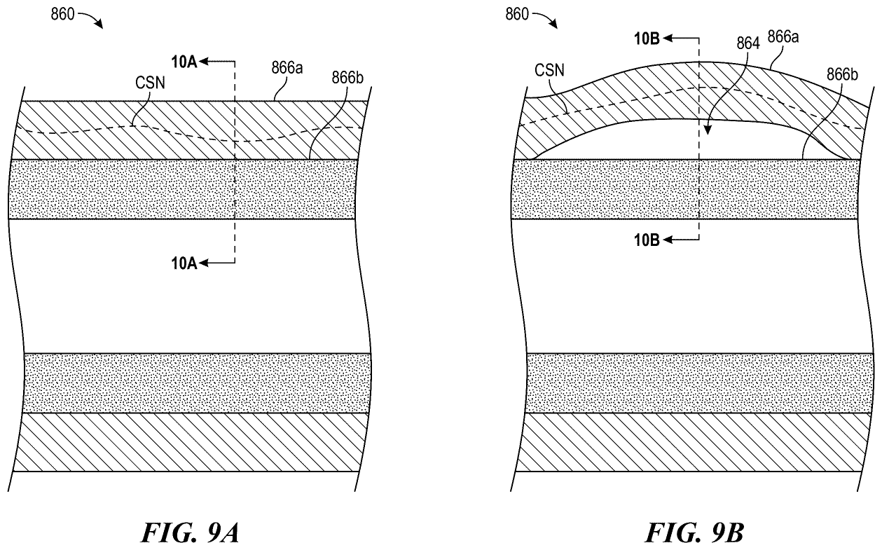

FIG. 9A is a cross-section taken along section line 9A-9A in FIG. 8A, and FIG. 9B is a cross-section taken along section linc 9B-9B in FIG. 8B. FIGS. 9A and 9B illustrate the opening 864 between the periadventitial tissue 866a and the ICA 866b, as described previously with reference to block 704. As described previously hercin, the ICA 866b can include intimal, medial, and adventitial tissues. The periadventitial tissue 866a can include the CSN and/or fibers associated with the baroreceptors, and surround the ICA 766b. Accordingly, a practitioner can separate the periadventitial tissue 866a from the ICA 866b to form the opening 864 without, or substantially without, penetrating into the adventitial, medial, and/or intimal tissues of the ICA 866b. The opening 864 enables a signal delivery device to be positioned on opposite sides of the target region 860 to deliver an electrical signal thereto, as described previously with reference to blocks 706-710 and FIGS. 8C and 8D.

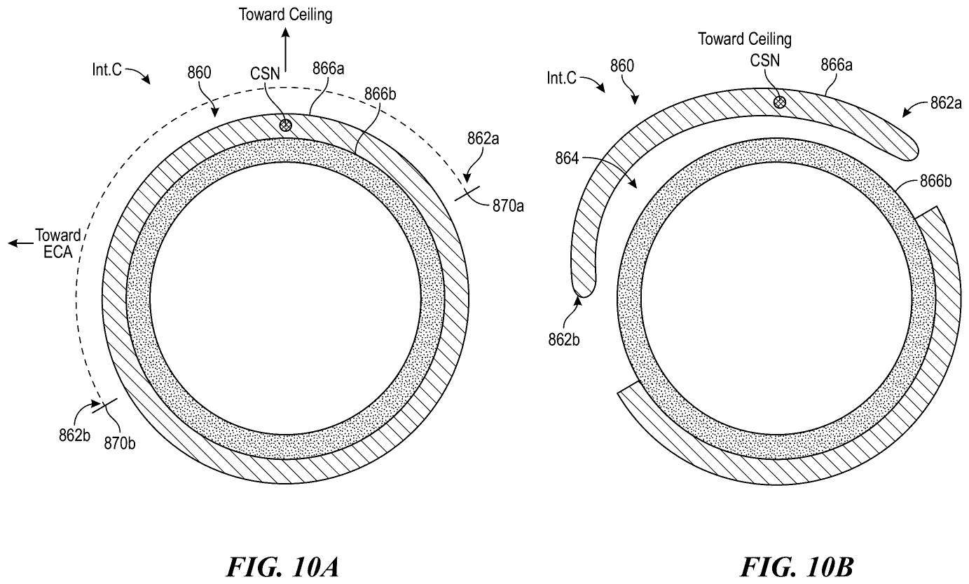

FIG. 10A is a cross-section taken along section line 10A-10A in FIG. 9A, and FIG. 10B is a cross-section taken along section line 10B-9B in FIG. 9B. As shown in FIG. 10A, the opening 864 can be formed between a first incision point 870a and a second incision point 870b. The first incision point 870a can be at the first side 862a of the target region 860 and/or approximately at a 2 o'clock position if using a clockface in which the 12 o'clock position points toward the ceiling. The second incision point 870b can be at or at least proximate to the second side 862b of the target region 860 and/or approximately at an 8 o'clock position if using the clockface. The CSN and/or the one or more fibers associated with the baroreceptors are expected to be located within this 8 o'clock to 2 o'clock range of the ICA, such that the CSN and/or the one or more fibers associated with the baroreceptors do not need to be specifically located when forming the opening 864. Accordingly, by forming the opening 864 between these two points, the practitioner can partially separate the periadventitial tissue 866a, including a section of the CSN and/or the one or more fibers associated with the baroreceptors, from the underlying ICA 866b, such as shown in FIG. 10B. In other embodiments, however, the practitioner can locate the CSN and/or the one or more fibers before, during, and/or after forming the opening 864, e.g., to confirm that the target region 860 and/or the separated section of the periadventitial tissue 866a includes the CSN and/or the one or more fibers. To form the opening 864, the practitioner can cut the target region 860 in a direction at least generally parallel to the longitudinal and/or circumferential axis of the ICA at the first side 862a and/or the second side 862b, e.g., to define a width of the opening 864, and then move the cutting tool in a curved or arcuate path about or at least partially around the longitudinal and/or circumferential axis of the ICA, generally following a circumferential border between the periadventitial tissue 866a and the underlying ICA 866b.

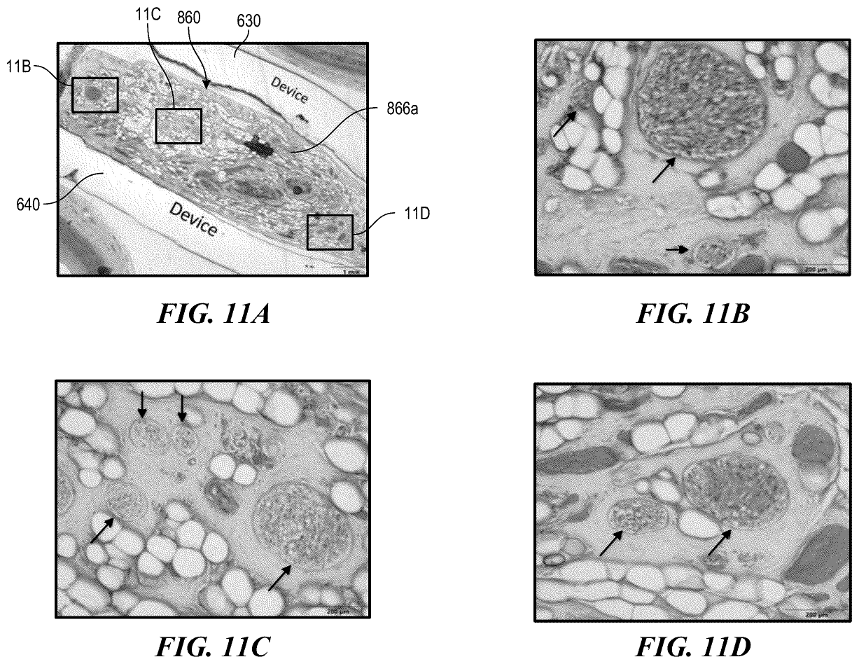

FIGS. 11A-11D are images of tissue illustrating representative implant positions for a signal delivery device, in accordance with embodiments of the present technology. Referring to FIG. 11A, the target region 860 can include multiple nerves, indicated by black arrows in FIGS. 11B-11D, in the periadventitial tissue 866a. At least one of these nerves is expected to include the CSN and/or one or more fibers associated with the baroreceptors. Accordingly, as described previously herein, positioning the periadventitial tissue 866a between the first and second regions 630, 640 of the signal delivery device enables the electrodes carried by the first and second regions 630, 640 to stimulate the CSN and/or one or more fibers associated with the baroreceptors, e.g., without first identifying these target neural structures.

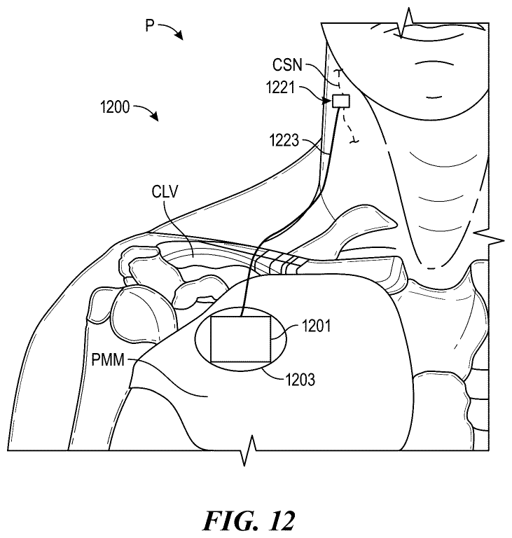

FIG. 12 is a partially schematic illustration of a patient treatment system 1200 (“system 1200”) implanted in a patient P, in accordance with embodiments, of the present technology. At least some elements of the system 1200 can be generally similar or identical in structure and/or function to the system 100 of FIGS. 3, 5A, and 5B. For example, the system 1200 includes an implantable neuromodulator 1201 (e.g., a signal generator or IPG) and one or more signal delivery elements or devices 1221 (“signal delivery device 1221”) electrically couplable to the neuromodulator 1201 via one or more conductors 1223. The neuromodulator 1201 can be at least generally similar or identical in structure and/or function to the implantable neuromodulator 101 of FIGS. 1A and 1B. The signal delivery device can be at least generally similar or identical to the signal delivery device 621 of FIGS. 6A and 6B. The conductors 1223 can be at least generally similar or identical in structure and/or function to the conductors 123 of FIGS. 1A and 1B.

The signal delivery device 1221 can be positioned to stimulate the CNS and/or one or more fibers associated with the baroreceptors, e.g., as described previously with reference to FIGS. 7-10B. The neuromodulator 1201 can be implanted inferior to the signal delivery device 1221, such as within a subcutaneous pocket 1203 located about 2-3 cm inferior to the patient's clavicle CLV and/or superficial to the fascia of the pectoralis major muscle (PMM). The subcutaneous pocket 1203 can be sized to have at least generally similar dimensions as the neuromodulator 1201, e.g., to provide a snug fit expected to reduce or prevent hematomas and/or migration of the neuromodulator 1201 after implantation. The conductors 1223 can be tunneled from the signal delivery device 1221 to the neuromodulator 1201, e.g., to electrically couple the signal delivery device 1221 to the neuromodulator 1201. The path of the conductors 1223 can remain anterior and/or superficial to the clavicle CLV. In some embodiments, the conductors 1223 can be implanted with at least a section of the conductors 1223 positioned in an S-shape or other arrangement configured to reduce, minimize, or prevent strain on the conductors 1223. In other embodiments, the neuromodulator 1201 can be miniaturized and/or otherwise configured for implantation in the patient's neck N.

FIG. 13 is a flow diagram of a method 1300 for positioning a signal delivery device, in accordance with embodiments of the present technology. The signal delivery device described herein can be one or more of the signal delivery devices 121, 621 described in FIGS. 5A-6B, or any other embodiments of the signal delivery devices described herein. Additionally or alternatively, the signal delivery device can be one or more components of the patient treatment systems described herein, such as the systems 100, 1200 described in FIGS. 3, 5A, 5B, and 12, or any other embodiments of the patient treatment systems described herein. The method 1300 is illustrated as a series of steps, process portions, or blocks 1302-1310. The method 1300 can be performed in combination with one or more steps, process portions, or blocks of the method 700 described in FIG. 7 and/or any of the other methods, steps, process portions, or blocks described herein.

At block 1302, the method 1300 can include identifying a target region of a blood vessel of the patient, as described in more detail with reference to block 702 of FIG. 7. In some embodiments, the target region is located superior to the carotid sinus (or the carotid bulb) of the patient. For example, the target region can be located no more than 0.5 cm, 1.5 cm, or 5 cm, within a range of 0.5 cm to 5 cm, or any value therebetween, superior to the carotid sinus of the patient. At block 1304, the method 1300 can include forming an opening at the target region to partially separate periadventitial tissue surrounding the blood vessel from the blood vessel, as described in more detail with reference to block 704 of FIG. 7. In some embodiments, the periadventitial tissue includes fibers associated with baroreceptors and/or the CSN of the patient. Additionally or alternatively, the blood vessel can be the ICA, and the fibers discussed herein can extend alongside or adjacent to the ICA. In some embodiments, forming the opening includes separating the periadventitial tissue from the adventitial, medial, and/or intimal tissues surrounding the ICA of the patient. In some embodiments, the opening is formed between a first side of the target region and a second side of the target region spaced apart from the first side.

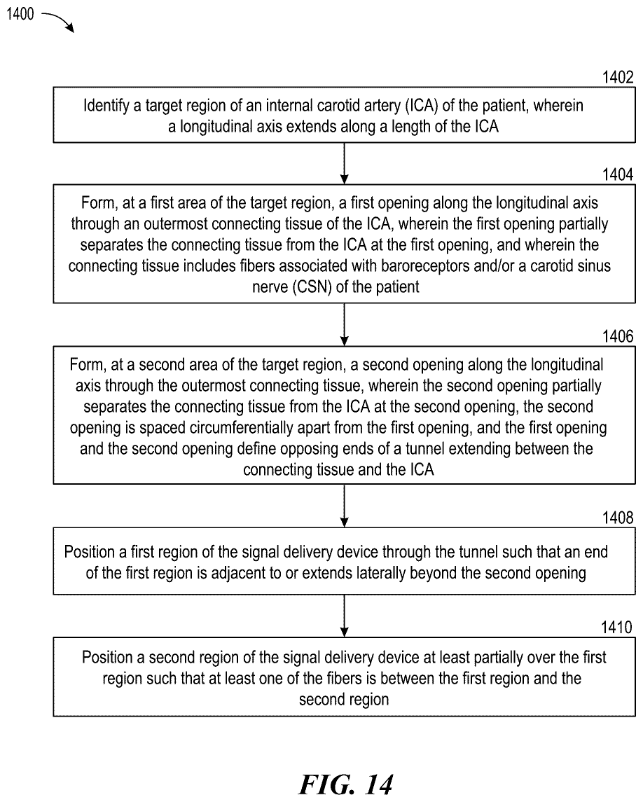

Additionally or alternatively, the opening can be formed between a first side of the target region and a second side of the target region spaced apart from the first side, with the separated periadventitial tissue remaining attached to the blood vessel except at the first opening, the second opening, and a tunnel between the first and second openings (e.g., the tunnel described in more detail with reference to block 1406 of FIG. 14). The opening can further be formed between a first side of the target region and a second side of the target region spaced apart from the first side, and the first region can be positioned such that a distal end of the first region extends through the opening beyond the second side of the target region. In some embodiments, the opening is formed by cutting around at least a portion of the circumference of the blood vessel. Additionally or alternatively, the opening can be formed by cutting in a direction at least generally parallel to the longitudinal axis of the blood vessel. In some embodiments, the size of the openings described herein are no more than 3 mm, 5 mm, 10 mm, within a range of 3 mm and 10 mm, or any value therebetween.

At block 1306, the method 1300 can include positioning a first region of the signal delivery device at least partially through the opening (e.g., the opening at the target region formed to partially separate periadventitial tissue surrounding the blood vessel described in more detail with reference to block 1304) such that the first region is between the blood vessel and the separated periadventitial tissue. The steps/process portions for positioning the first region of the signal delivery device are described in more detail with reference to block 706 of FIG. 7. In some embodiments, positioning the first region of the signal delivery device includes positioning one or more first electrodes of a signal delivery device at least partially through the opening.

As shown in FIG. 13, at block 1308, the method 1300 can include positioning a second region of the signal delivery device at least partially over the first region such that the separated periadventitial tissue is between the first region and the second region, as described in more detail with reference to block 708 of FIG. 7. In some embodiments, positioning the second region at least partially over the first region involves positioning the second region directly over the fibers associated with baroreceptors, the vagus nerve, and/or the CSN of the patient. Additionally or alternatively, the second region can be positioned at least partially over the first region by folding the second region at least partially over the first region. In some embodiments, the first region and the second region have respective ends that are coupled to a third region of the signal delivery device, and the second region is positioned at least partially over the first region by moving the second region relative to the first region about the third region. Additionally or alternatively, after positioning the second region of the signal delivery device at least partially over the first region, a first end of the first region can be coupled or sutured to a second end of the second region. In some embodiments, rather than by forming one or more openings at the target region, the target region can be identified based on the methods described herein, and the signal delivery device can be positioned to envelop or abut the target region and/or affixed onto the target region in one or more alternative manners compared to the embodiment described above.

In some embodiments, positioning the second region of the signal delivery device can include positioning one or more second electrodes of a signal delivery device at least partially over the first region. After positioning the second region over the first region, an electrical signal can be delivered to the separated periadventitial tissue between the first region and the second region via the one or more first electrodes and/or the one or more second electrodes. Although not explicitly called out in a block, the method 1300 can further include providing stimulation (e.g., to the nerves and/or fibers within the separated surrounding tissue) via one or more electrodes of the signal delivery device, which is described in more detail with reference to block 710 of FIG. 7. For example, stimulation can include an electrical signal provided to the fibers of the patient. In some embodiments, a pulse generator is implanted inferior to the signal delivery device. The pulse generator can be electrically coupled to the signal delivery device and configured to generate one or more of the electrical signals described herein.

In some embodiments, a first electrode of the signal delivery device is selected to deliver a first electrical signal to the patient. The first electrical signal can be delivered via the first electrode, and the patient's response to the electrical signal can be recorded to, for example, determine or verify the positioning of the signal delivery device. In some embodiments, direct visualization and/or one or more external sensors, devices, or systems are used to determine whether the patient's response to the electrical signal is less than, greater than, or within a response threshold, as described in more detail with reference to FIGS. 3 and 4. If the patient's response is determined to be less than the response threshold, a second electrical signal can be delivered via a second electrode, different from the first electrode, of the signal delivery device, and the patient's response to the second electrical signal can be determined.

In some embodiments, a first value of a signal delivery parameter is selected at which the signal delivery device delivers an electrical signal to the patient (e.g., via the first electrode). The electrical signal can be delivered at the first value, and it can be determined that the patient's response to the electrical signal is less than a response threshold. If the patient's response is less than the response threshold, the electrical signal can be delivered at a second value of the signal delivery parameter (e.g., via the second electrode), different from the first value, and the patient's response can be determined once again. Any number of the electrodes can be repositioned one or more times, and the process repeated if the patient's response remains below the threshold response. If the patient's response to the second electrode is determined to be equal to or greater than the response threshold, a therapy signal (e.g., stimulation) can be delivered to the patient via the second electrode of the signal delivery device.

In some embodiments, the patient response involves a change in the blood pressure of the patient. More specifically, the patient response can involve a reduction in the patient's blood pressure and determining that the patient response is less than the response threshold can include determining that the reduction is less than 5 mmHg, 10 mmHg, or 40 mmHg, within a range of 5 mmHg to 40 mmHg, or any value therebetween. Additionally or alternatively, the patient response can involve a reduction in the patient's blood pressure, and determining that the patient response is less than the response threshold can include determining that the reduction in the patient's blood pressure is less than 2.5%, 5%, or 20%, within a range of 2.5% to 20%, or any value therebetween. In some embodiments, determining the patient's response involves assessing a change in the heart rate of the patient. For example, the patient response can be considered less than the response threshold if the change in the patient's heart rate is less than 2.5%, 5%, 10%, 20%, or 40%, within a range of 2.5% to 40%, or any value therebetween.

FIG. 14 is a flow diagram of a method 1400 for positioning a signal delivery device, in accordance with embodiments of the present technology. The signal delivery device described herein can be one or more of the signal delivery devices 121, 621 described in FIGS. 5A-6B, or any other embodiments of the signal delivery devices described herein. Additionally or alternatively, the signal delivery device can be one or more components of the patient treatment systems described herein, such as the systems 100, 1200 described in FIGS. 3, 5A, 5B, and 12, or any other embodiments of the patient treatment system described herein. The method 1400 is illustrated as a series of steps, process portions, or blocks 1402-1410. The method 1400 can be performed in combination with one or more steps, process portions, or blocks of the methods 700, 1300 described in FIGS. 7 and 13, respectively, and/or any of the other methods, steps, process portions, or blocks described herein.

At block 1402, the method 1400 can include identifying a target region of an ICA of a patient. In some embodiments, a longitudinal axis that extends along the length of the ICA can be identified using direct visualization and/or one or more imaging modalities such as ultrasound or OCT to confirm that the target region includes the ICA and to prepare the region for incisions. Additionally or alternatively, the ICA can be identified using one or more of the steps, processes, or blocks used to identify a blood vessel of the patient, as described in more detail with reference to blocks 702 and 1302 of FIGS. 7 and 13, respectively. For example, as described in more detail with reference to FIG. 7, identifying the target region can include identifying a circumference of the connecting tissue of the ICA expected to include the CSN and/or one or more fibers associated with the baroreceptors. The circumference expected to include the CSN can be noted by the physician when identifying the longitudinal axis along the length of the ICA used to create the openings for positioning the patient treatment system, as described in more detail below. In some embodiments, the target region includes the entirety of the connecting tissue between the ICA and an external carotid artery (ECA) of the patient such that the signal delivery device can be positioned to envelop both the tissue surrounding and/or between the ICA and the ECA.

At block 1404, the method 1400 can include forming, at a first area of the target region, a first opening along the longitudinal axis through the outermost connecting tissue of the ICA. The process for forming an opening at the first area of the target region can be identical or generally similar to the process of forming an opening at a side of the target region, as described in more detail with reference to blocks 704 and 1304 of FIGS. 7 and 13, respectively. In some embodiments, the first opening separates the connecting tissue that includes the CSN and/or fibers associated with the baroreceptors from the ICA at the first area of the target region. The first opening can be formed using one or more tools such blunt dissectors (e.g. nerve dissector or right-angled dissecting forceps) or sharp dissectors (e.g. such as a scalpel or surgical scissors). In some embodiments, a physician identifies where to create the first opening. Additionally or alternatively, one or more external systems described herein can be used in place of or with the physician to identify where to form the first opening, such as the external systems (e.g., the external device 111 and CSNS neuromonitoring system 400) described in more detail with reference to FIGS. 3-5B.

At block 1406, the method 1400 can include forming, at a second area of the target region, a second opening along the longitudinal axis through the outermost connecting tissue. The process for forming the second opening at the second area of the target region can be identical or generally similar to the process of forming the first opening at the first area of the target region, as described in more detail with reference to block 1404. In some embodiments, the second opening at least partially separates the connecting tissue from the ICA at the second opening. As described at block 1402, when identifying the longitudinal axis along the length of the ICA, a circumference expected to include the CSN can be identified. The circumference expected to include the CSN can be used to identify the first and second areas of the target region and thus the position of the first and second openings. For example, the second opening can be spaced circumferentially apart from the first opening. Additionally or alternatively, the first opening and the second opening can define opposing ends of a tunnel extending between the connecting tissue and the ICA used to position the patient treatment system described herein. In some embodiments, the method 1400 further includes identifying the ECA in the target area of the patient and a second longitudinal axis extending along the length of the ECA for forming an opening. The method 1400 can also include forming an opening (e.g., a second or third opening) along the second longitudinal axis through the connecting tissue (e.g., the innermost and outermost tissue) of the ECA and extending this opening to an opening along the ICA (e.g., the first or second openings described above).

At block 1408, the method 1400 can include positioning a first region of a signal delivery device through a tunnel (e.g., the tunnel extending between the connecting tissue and the ICA described in more detail with reference to block 1406) such that an end of the first region of the signal delivery device is adjacent to or extends laterally beyond the second opening. The steps/process portions for positioning the first region of the signal delivery device are described in more detail with reference to blocks 706 and 1308 of FIGS. 7 and 13, respectively. As shown in FIG. 14 at block 1410, the method 1400 can include positioning a second region of the signal delivery device at least partially over the first region such that at least one of the fibers is between the first region and the second region, as described in more detail with reference to blocks 708 and 1310 of FIGS. 7 and 13, respectively. The method 1400 can further comprise positioning a first and/or a second region of the signal delivery device such that at least a portion of both the ICA and the ECA is enveloped. Although not explicitly called out in a block, the method 1400 can further include providing stimulation (e.g., to the nerves and/or fibers within the separated surrounding tissue) via one or more electrodes of the signal delivery device, which is described in more detail with reference to block 710 of FIG. 7.

### IV. Conclusion

It will be apparent to those having skill in the art that changes may be made to the details of the above-described embodiments without departing from the underlying principles of the present disclosure. In some cases, well known structures and functions have not been shown or described in detail to avoid unnecessarily obscuring the description of the embodiments of the present technology. Although steps of methods may be presented herein in a particular order, alternative embodiments may perform the steps in a different order. Similarly, certain aspects of the present technology disclosed in the context of particular embodiments can be combined or eliminated in other embodiments. Furthermore, while advantages associated with certain embodiments of the present technology may have been disclosed in the context of those embodiments, other embodiments can also exhibit such advantages, and not all embodiments need necessarily exhibit such advantages or other advantages disclosed herein to fall within the scope of the technology. Accordingly, the disclosure and associated technology can encompass other embodiments not expressly shown or described herein, and the invention is not limited except as by the appended claims.

Throughout this disclosure, the singular terms “a,” “an,” and “the” include plural referents unless the context clearly indicates otherwise. Additionally, the term “comprising,” “including,” and “having” should be interpreted to mean including at least the recited feature(s) such that any greater number of the same feature and/or additional types of other features are not precluded.

Reference herein to “one embodiment,” “an embodiment,” “some embodiments,” or similar formulations means that a particular feature, structure, operation, or characteristic described in connection with the embodiment can be included in at least one embodiment of the present technology. Thus, the appearances of such phrases or formulations herein are not necessarily all referring to the same embodiment. Furthermore, various particular features, structures, operations, or characteristics may be combined in any suitable manner in one or more embodiments.

Unless otherwise indicated, all numbers expressing pressures, frequencies, amplitudes, duty cycles, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present technology. At the very least, and not as an attempt to limit the application of the doctrine of equivalents to the scope of the claims, each numerical parameter should at least be construed in light of the number of reported significant digits and by applying ordinary rounding techniques. Additionally, all ranges disclosed herein are to be understood to encompass any and all subranges subsumed therein. For example, a range of “1 to 10” includes any and all subranges between (and including) the minimum value of 1 and the maximum value of 10 (i.e., any and all subranges having a minimum value of equal to or greater than 1 and a maximum value of equal to or less than 10 (e.g., 5.5 to 10)).

The disclosure set forth above is not to be interpreted as reflecting an intention that any claim requires more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

The present technology is illustrated, for example, according to various aspects described below as numbered clauses (1, 2, 3, etc.) for convenience. These are provided as examples and do not limit the present technology. It is noted that any of the dependent clauses may be combined in any combination, and placed into a respective independent clause.

1. A method of implanting a signal delivery device to deliver an electrical signal to a patient, the method comprising:
identifying a target region of a blood vessel of the patient, wherein the target region is located superior to a carotid sinus of the patient;
forming an opening at the target region to partially separate periadventitial tissue surrounding the blood vessel from the blood vessel, wherein the periadventitial tissue includes fibers associated with baroreceptors and/or a carotid sinus nerve (CSN) of the patient;
positioning a first region of the signal delivery device at least partially through the opening such that the first region is between the blood vessel and the separated periadventitial tissue; and
positioning a second region of the signal delivery device at least partially over the first region such that the separated periadventitial tissue is between the first region and the second region.

2. The method of any one of the clauses herein, wherein the blood vessel is an internal carotid artery (ICA) and the fibers extend alongside or adjacent the ICA.

3. The method of any one of the clauses herein, wherein positioning the second region at least partially over the first region includes positioning the second region directly over the fibers associated with baroreceptors, vagus nerve, and/or the CSN of the patient.

4. The method of any one of the clauses herein, wherein:
the first region includes one or more first electrodes,
the second region includes one or more second electrodes, and
the method further comprises, after positioning the second region over the first region, delivering the electrical signal to the separated periadventitial tissue between the first region and the second region via the one or more first electrodes and/or the one or more second electrodes.

5. The method of clause 4, wherein delivering the electrical signal includes delivering the electrical signal to the fibers of the patient.

6. The method of any one of the clauses herein, wherein identifying the target region includes identifying the target region superior to a carotid sinus of the patient.

7. The method of any one of the clauses herein, wherein identifying the target region includes identifying the target region no more than 1 centimeter (cm), 1.5 cm, or 2 cm superior to a carotid sinus of the patient.

8. The method of any one of the clauses herein, wherein identifying the target region includes identifying the target region no more than 2 centimeters (cm) superior to a carotid sinus of the patient.

9. The method of any one of the clauses herein, wherein identifying the target region includes identifying the target region between 1 centimeter (cm) and 2 cm superior to a carotid sinus of the patient.

10. The method of any one of the clauses herein, wherein forming the opening includes separating the periadventitial tissue from adventitial, medial, and/or intimal tissues surrounding an internal carotid artery (ICA) of the patient.

11. The method of any one of the clauses herein, wherein the opening is no more than 10 millimeters (mm), 5 mm, or 3 mm.

12. The method of any one of the clauses herein, wherein the opening is no more than 10 millimeters (mm).

13. The method of any one of the clauses herein, wherein the opening is between 3 millimeters (mm) and 10 mm.

14. The method of any one of the clauses herein, wherein forming the opening includes forming the opening between a first side of the target region and a second side of the target region spaced apart from the first side.

15. The method of any one of the clauses herein, wherein forming the opening includes forming the opening between a first side of the target region and a second side of the target region spaced apart from the first side, wherein the separated periadventitial tissue remains attached to the blood vessel except at the opening and a tunnel between the opening.

16. The method of any one of the clauses herein, wherein forming the opening includes forming the opening between a first side of the target region and a second side of the target region spaced apart from the first side, wherein positioning the first region comprises positioning the first region such that a distal end of the first region extends through the opening beyond the second side of the target region.

17. The method of any one of the clauses herein, wherein forming the opening includes cutting around at least a portion of a circumference of the blood vessel.

18. The method of any one of the clauses herein, wherein forming the opening includes cutting in a direction at least generally parallel to a longitudinal axis of the blood vessel.

19. The method of any one of the clauses herein, further comprising:
delivering one or more electrical signals via one or more electrodes;
determining a patient response to the one or more electrical signals; and
modifying a position of the one or more electrodes based on a comparison of the patient response to a response threshold.

20 The method of clause 19, wherein the patient response is a hemodynamic response.

21. The method of clause 19, further comprising:
processing the patient response using one or more calibration techniques to eliminate noise.

22. The method of clause 19, wherein the one or more electrodes are electrically decoupled from the signal delivery device.

23. The method of clause 19, wherein the one or more electrodes are arranged in at least one of a spiral cuff arrangement, a flat cuff arrangement, or a single surface array/electrode arrangement.

24. The method of claim 1, wherein the signal delivery device comprises a spiral cuff including two or more electrodes, and wherein the spiral cuff surrounds the opening and at least a portion of the blood vessel.

25. The method of clause 19, further comprising:
repositioning the one or more electrodes based on the comparison of the patient response to the response threshold;
delivering one or more additional electrical signals via one or more repositioned electrodes;
determining an additional patient response to the one or more additional electrical signals;
in response to a determination that the additional patient response is less than the response threshold, modifying the position of the one or more repositioned electrodes; and
in response to a determination that the additional patient response is greater than or equal to the response threshold, delivering a therapy signal to the patient via the one or more repositioned electrodes.

26. The method of clause 19, further comprising:
displaying the patient response; and
displaying the response threshold relative to the patient response to visualize a comparison.

27. The method of clause 26, wherein displaying the patient response includes displaying one or more signal sweeps representative of physiological parameters of the patient during the delivery of electrical signals.

28. The method of clause 26, wherein displaying the patient response includes displaying one or more signal sweeps representative of physiological parameters of the patient during activation and deactivation periods of delivering electrical signals.

29. The method of any one of the clauses herein, further comprising:
selecting a first electrode of the signal delivery device to deliver a first electrical signal to the patient;
delivering the first electrical signal via the first electrode;
determining that a patient response to the electrical signal is less than a response threshold; and
after determining the patient response, delivering a second electrical signal via a second electrode of the signal delivery device different than the first electrode.

30. The method of clause 29, further comprising:
determining that the patient response to the second electrical signal is equal to or greater than the response threshold; and
delivering a therapy signal to the patient via the second electrode.

31. The method of any one of the clauses herein, further comprising:
selecting a first value of a signal delivery parameter at which the signal delivery device delivers an electrical signal to the patient;
delivering the electrical signal at the first value;
determining that a patient response to the electrical signal is less than a response threshold, and
after determining the patient response, delivering the electrical signal at a second value of the signal delivery parameter, different than the first value.

32. The method of clause 29, wherein the patient response includes a change to a blood pressure of the patient.

33. The method of clause 29 or clause 32, wherein the patient response includes a reduction in the patient's blood pressure and wherein determining that the patient response is less than the response threshold includes determining that a reduction in the blood pressure of the patient is less than 5 mmHg, 10 mmHg, or 40 mmHg.

34. The method of clause 29 or clause 32, wherein the patient response includes a reduction in the patient's blood pressure and wherein determining that the patient response is less than the response threshold includes determining that a reduction in the blood pressure of the patient is less than 10 mmHg.

35. The method of clause 29 or clause 32, wherein the patient response includes a reduction in the patient's blood pressure and wherein determining that the patient response is less than the response threshold includes determining that a reduction in the blood pressure of the patient is between 5 mmHg and 40 mmHg.

36. The method of any one of clauses 29-35, wherein the patient response includes a reduction in the patient's blood pressure and wherein determining that the patient response is less than the response threshold includes determining that a reduction in the blood pressure of the patient is less than 5%, 10%, or 20%.

37. The method of any one of clauses 29-35, wherein the patient response includes a reduction in the patient's blood pressure and wherein determining that the patient response is less than the response threshold includes determining that a reduction in the blood pressure of the patient is less than 5%.

38. The method of any one of clauses 29-35, wherein the patient response includes a reduction in the patient's blood pressure and wherein determining that the patient response is less than the response threshold includes determining that a reduction in the blood pressure of the patient is between 5% and 20%.

39 The method of any one of clauses, wherein the patient response includes a change in a heart rate of the patient and wherein determining that the patient response is less than the response threshold includes determining that the change in the heart rate of the patient is less than 5%, 10%, 20%, or 40%.

40. The method of any one of clauses, wherein the patient response includes a change in a heart rate of the patient and wherein determining that the patient response is less than the response threshold includes determining that the change in the heart rate of the patient is less than 5% or 10%.

41 The method of any one of clauses, wherein the patient response includes a change in a heart rate of the patient and wherein determining that the patient response is less than the response threshold includes determining that the change in the heart rate of the patient is between 5% and 40%.

42. The method of any one of the clauses herein, further comprising:
implanting a pulse generator inferior to the signal delivery device, wherein the pulse generator is configured to generate one or more electrical signals; and
electrically coupling the signal delivery device to the pulse generator.

43. The method of any one of the clauses herein, wherein positioning the second region at least partially over the first region includes folding the second region at least partially over the first region.

44. The method of any one of the clauses herein, wherein the first region and the second region include respective ends that are coupled to a third region of the signal delivery device, and wherein positioning the second region at least partially over the first region includes moving the second region relative to the first region about the third region.

45. The method of any one of the clauses herein, further comprising, after positioning the second region of the signal delivery device at least partially over the first region, coupling or suturing a first end of the first region to a second end of the second region.

46. The method of clause 45, wherein coupling the first and second ends of the first and second regions includes tying a suture between the first and second ends.

47. The method of clause 45, wherein coupling the first and second ends of the first and second regions includes coupling the first and second ends with a vessel clip.

48 The method of any one of the clauses herein, wherein identifying the target region of the blood vessel comprises using ultrasound or optical coherence tomography (OCT) to identify the target region.

49. A method of implanting a signal delivery device to deliver an electrical signal to a patient, the method comprising:
identifying a target region of an internal carotid artery (ICA) of the patient, wherein a longitudinal axis extends along a length of the ICA;
forming, at a first area of the target region, a first opening along the longitudinal axis through an outermost portion of a connecting tissue of the ICA, wherein the first opening partially separates the connecting tissue from the ICA at the first opening, and wherein the connecting tissue includes fibers associated with baroreceptors and/or a carotid sinus nerve (CSN) of the patient;
forming, at a second area of the target region, a second opening along the longitudinal axis through the outermost connecting tissue, wherein:
   the second opening partially separates the connecting tissue from the ICA at the second opening,
   the second opening is spaced circumferentially apart from the first opening, and
   the first opening and the second opening define opposing ends of a tunnel extending between the connecting tissue and the ICA;
positioning a first region of the signal delivery device through the tunnel such that an end of the first region is adjacent to or extends laterally beyond the second opening; and
positioning a second region of the signal delivery device at least partially over the first region such that at least one of the fibers is between the first region and the second region.

50. The method of any one of the clauses herein, wherein the target region includes an entirety of the connecting tissue between the ICA and an external carotid artery (ECA) of the patient.

51. The method of any one of the clauses herein, wherein the target region includes an entirety of the connecting tissue between the ICA and an external carotid artery (ECA) of the patient, the method further comprising:
positioning the first and/or the second region of the signal delivery device such that at least a portion of tissue surrounding both the ICA and the ECA is enveloped.

52. The method of any one of the clauses herein, wherein the longitudinal axis is a first longitudinal axis, the method further comprising:
identifying an external carotid artery (ECA) of the patient, wherein the ECA is within the target region, and wherein a second longitudinal axis extends along a length of the ECA;
forming, a third opening along the second longitudinal axis through an outermost connecting tissue of the ECA; and
extending the third opening to the second opening along the ICA.

1. A method of implanting a signal delivery device to deliver an electrical signal to a patient, the method comprising:
identifying a target region of a blood vessel of the patient, wherein the target region is located superior to a carotid sinus of the patient;
forming an opening at the target region to partially separate periadventitial tissue surrounding the blood vessel from the blood vessel, wherein the periadventitial tissue includes fibers associated with baroreceptors and/or a carotid sinus nerve (CSN) of the patient;
positioning a first region of the signal delivery device at least partially through the opening such that the first region is between the blood vessel and the separated periadventitial tissue; and
positioning a second region of the signal delivery device at least partially over the first region such that the separated periadventitial tissue is between the first region and the second region.

2. The method of claim 1, wherein the blood vessel is an internal carotid artery (ICA) and the fibers extend alongside or adjacent the ICA.

3. The method of claim 1, wherein positioning the second region at least partially over the first region includes positioning the second region directly over the fibers associated with the baroreceptors, vagus nerve, and/or the CSN of the patient.

4. The method of claim 1, wherein:
the first region includes one or more first electrodes,
the second region includes one or more second electrodes, and
the method further comprises, after positioning the second region over the first region, delivering the electrical signal to the separated periadventitial tissue between the first region and the second region via the one or more first electrodes and/or the one or more second electrodes.

5. The method of claim 4, wherein delivering the electrical signal includes delivering the electrical signal to the fibers of the patient.

6. The method of claim 1, wherein identifying the target region includes identifying the target region superior to a carotid sinus of the patient.

7. The method of claim 1, wherein identifying the target region includes identifying the target region no more than 2 centimeters (cm) superior to a carotid sinus of the patient.

8. The method of claim 1, wherein forming the opening includes separating the periadventitial tissue from adventitial, medial, and/or intimal tissues surrounding an internal carotid artery (ICA) of the patient.

9. The method of claim 1, wherein the opening is no more than 10 millimeters (mm).

10. The method of claim 1, wherein forming the opening includes forming the opening between a first side of the target region and a second side of the target region spaced apart from the first side.

11. The method of claim 1, wherein forming the opening includes forming the opening between a first side of the target region and a second side of the target region spaced apart from the first side, wherein the separated periadventitial tissue remains attached to the blood vessel except at the opening and a tunnel between the opening.

12. The method of claim 1, wherein forming the opening includes forming the opening between a first side of the target region and a second side of the target region spaced apart from the first side, wherein positioning the first region comprises positioning the first region such that a distal end of the first region extends through the opening beyond the second side of the target region.

13. The method of claim 1, further comprising:
delivering one or more electrical signals via one or more electrodes;
determining a patient response to the one or more electrical signals; and
modifying a position of the one or more electrodes based on a comparison of the patient response to a response threshold.

14. The method of claim 13, further comprising:
displaying the patient response; and
displaying the response threshold relative to the patient response to visualize a comparison.

15. The method of claim 1, wherein the signal delivery device comprises a spiral cuff including two or more electrodes, and wherein the spiral cuff surrounds the opening and at least a portion of the blood vessel.

16. The method of claim 1, further comprising:
selecting a first electrode of the signal delivery device to deliver a first electrical signal to the patient;
delivering the first electrical signal via the first electrode;
determining that a patient response to the electrical signal is less than a response threshold; and
after determining the patient response, delivering a second electrical signal via a second electrode of the signal delivery device different than the first electrode.

17. The method of claim 16, further comprising:
determining that the patient response to the second electrical signal is equal to or greater than the response threshold; and
delivering a therapy signal to the patient via the second electrode.

18. The method of claim 17, wherein the patient response includes a change to a blood pressure of the patient.

19. The method of claim 18, wherein determining that the patient response is less than the response threshold includes determining that a reduction in the blood pressure of the patient is less than 10 mmHg.

20. The method of claim 18, wherein determining that the patient response is less than the response threshold includes determining that a reduction in the blood pressure of the patient is less than 5%.

21. The method of claim 16, wherein the patient response includes a change in a heart rate of the patient and wherein determining that the patient response is less than the response threshold includes determining that the change in the heart rate of the patient is less than 5% or 10%.

22. The method of claim 1, further comprising:
implanting a pulse generator inferior to the signal delivery device, wherein the pulse generator is configured to generate one or more electrical signals; and
electrically coupling the signal delivery device to the pulse generator.

23. The method of claim 1, wherein positioning the second region at least partially over the first region includes folding the second region at least partially over the first region.

24. The method of claim 1, wherein the first region and the second region include respective ends that are coupled to a third region of the signal delivery device, and wherein positioning the second region at least partially over the first region includes moving the second region relative to the first region about the third region.

25. The method of claim 1, further comprising, after positioning the second region of the signal delivery device at least partially over the first region, coupling or suturing a first end of the first region to a second end of the second region.

26. The method of claim 1, wherein identifying the target region of the blood vessel comprises using ultrasound or optical coherence tomography (OCT) to identify the target region.

27. A method of implanting a signal delivery device to deliver an electrical signal to a patient, the method comprising:
identifying a target region of an internal carotid artery (ICA) of the patient, wherein a longitudinal axis extends along a length of the ICA;
forming, at a first area of the target region, a first opening along the longitudinal axis through an outermost portion of a connecting tissue of the ICA, wherein the first opening partially separates the connecting tissue from the ICA at the first opening, and wherein the connecting tissue includes fibers associated with baroreceptors and/or a carotid sinus nerve (CSN) of the patient;
forming, at a second area of the target region, a second opening along the longitudinal axis through the outermost connecting tissue, wherein:
the second opening partially separates the connecting tissue from the ICA at the second opening,
the second opening is spaced circumferentially apart from the first opening, and
the first opening and the second opening define opposing ends of a tunnel extending between the connecting tissue and the ICA;
positioning a first region of the signal delivery device through the tunnel such that an end of the first region is adjacent to or extends laterally beyond the second opening; and
positioning a second region of the signal delivery device at least partially over the first region such that at least one of the fibers is between the first region and the second region.

28. The method of claim 27, wherein the target region includes an entirety of the connecting tissue between the ICA and an external carotid artery (ECA) of the patient.

29. The method of claim 27, wherein the target region includes an entirety of the connecting tissue between the ICA and an external carotid artery (ECA) of the patient, the method further comprising:
positioning the first and/or the second region of the signal delivery device such that at least a portion of tissue surrounding both the ICA and the ECA is enveloped.

30. The method of claim 27, wherein the longitudinal axis is a first longitudinal axis, the method further comprising:
identifying an external carotid artery (ECA) of the patient, wherein the ECA is within the target region, and wherein a second longitudinal axis extends along a length of the ECA;
forming, a third opening along the second longitudinal axis through an outermost connecting tissue of the ECA; and
extending the third opening to the second opening along the ICA.
